(19) United States
(12) Patent Application Publication
(10) Pub. No.: US 2026/0215953 A1
(43) Pub. Date: Jul. 30, 2026

(54) PRE-FORMED ABSORBENT WOUND DRESSING, COMPRESSION SYSTEM AND METHODS OF USE

(71) Applicant: Sun Scientific, Inc., Dobbs Ferry, NY (US)

(72) Inventors: Sundaram Ravikumar, Braircliff Manor, NY (US); Vikram Ravikumar, New York, NY (US); Guy Osborne, Trumbull, CT (US); Harry Allan Alward, Shelton, CT (US)

(73) Assignee: Sun Scientific, Inc., Dobbs Ferry, NY (US)

(21) Appl. No.: 19/145,751

(22) PCT Filed: Aug. 15, 2024
(86) PCT No.: PCT/US2024/042582

§ 371 Date: Jul. 3, 2025

Related U.S. Application Data
(63) Continuation of application No. PCT/US2024/010513, filed on Jan. 5, 2024.
(60) Provisional application No. 63/526,349, filed on Jul. 12, 2023.

(51) Int. Cl. A61F 5/30; A61L 15/42; A61L 15/52

(52) U.S. Cl. CPC A61F 5/30; A61L 15/425; A61L 15/52

(57) ABSTRACT

A pre-formed absorbent wound dressing including at least three layers of a porous hydrophilic wound contact layer, an absorbent layer, and a hydrophobic moisture transfer layer, as well as an optional wicking layer. The pre-formed absorbent wound dressing is shaped and pre-formed configured for application on a selected or target body part such as a foot, ankle, leg, calf, knee, thigh, hip, torso, chest, shoulder, arm, elbow, wrist, hand, neck, and head. An inventive compression system includes the pre-formed absorbent wound dressing under a therapeutic compression apparatus.

### RELATED APPLICATIONS

The present application claims priority from U.S. provisional application Ser. No. 63/437,267 filed Jan. 5, 2023 entitled “Pre-formed Absorbent Wound Dressing, Therapeutic Compression System, and Methods of Use”, and U.S. provisional application Ser. No. 63/460,848 filed Apr. 20, 2023 entitled “Wound Dressing Compression System Including Pre-formed Absorbent Wound Dressing and Therapeutic Compression Garment and Methods of Use”, and U.S. provisional application Ser. No. 63/462,499 filed Apr. 27, 2023 entitled “Absorbent Foot Wound Dressing with Toe Spacers” and U.S. provisional application Ser. No. 63/526,349 filed Jul. 12 2023 entitled “Wound Dressing Compression System Including Pre-formed Absorbent Wound Dressing and Methods of Use”, and PCT/US2024/010513 entitled “Pre-formed Absorbent Wound Dressing, Therapeutic Compression System, and Methods of Use” filed Jan. 5, 2024, and PCT/US2024/042582 entitled “Pre-formed Absorbent Wound Dressing, Therapeutic Compression System, and Methods of Use” filed Aug. 15, 2024, the entire contents of which are hereby incorporated herein by reference for all purposes.

### BACKGROUND OF THE INVENTION

### I. Field of the Invention

The subject invention is directed generally to wound dressings and more particularly to a pre-formed absorbent wound dressing, as well as a system for applying compression over the inventive pre-formed absorbent wound dressing and more particularly, to a system for applying compression to the foot, ankle, calf, lower leg, thigh, full leg, hip, torso, chest, shoulder, arm, elbow, wrist, hand, neck, head or any body part of an individual in conjunction with a bolster dressing, elevation members, or another dressing with a medication imbedded within it, a pre-formed wound dressing, both under a compression wrap for use to treat a wound or in the treatment of conditions such as ulcers, vein thrombosis (DVT), chronic venous insufficiency (CVI) and lymphedema.

### II. Background of the Related Art

Wound dressings for centuries has concentrated on cotton or gauze bandages that absorb exudate from a wound, but then have the problem of leakage onto the user's clothes, bedsheets, furniture and other objects. There are a number of tapes and bandages that are available for application to a wound site or ulcer but they require a skilled medical professional to apply in rolled form onto the target body part with enough tension to keep the wound dressing on the target body part as the person goes about their daily life with walking, moving arms, head, and other body parts. Further, in cases of emergency the medical professional needs a wound dressing that can be applied immediately to the wound site without the time and skill of rolling bandages and wound dressings on the target body part. The anatomy and structure of the foot, leg, though, hip, torso, arm, shoulder, head and other body parts cause problems with adhesion and pressure of known wound dressings complicating any treatment and healing, as well as leakage or exudating of the wound site onto the individual's sock, clothing, bedsheets, couch and the like.

Wounds or ulcers on the foot are typically hard to treat due to the structure of the foot and fact that the foot is moved and under pressure when walking. Conventional bandages do not adhere to the foot, either on the top of the arch or below on the sole of the foot. Further, the foot area may become hot and moist contributing to lack of adhesion yet a diabetic foot may be extremely dry and the adhesive must be chosen not to irritate or affect the dry skin of for example a diabetic individual. The structure of the foot is also problematic in keeping a bandage or wound dressing in place and adhered to the foot. On the other side of the body any wound or ulcer on the head, face, scalp or neck is likewise hard to treat due to the structure of the head and bleed factors due to the structure of skin over the skull and bony prominences of the head. Any wound or ulcer bleeds more on the foot or head or for example elbow of hand than a similar wound or ulcer on other body parts. A need exists for a pre-formed absorbent wound dressing for body parts to address wounds, exudate and application to the target body part at initial dressing as well as being able to open and check on the wound after application while being able to reclose said wound dressing by the individual themselves without a skilled medical provider.

Diabetic ulcers may affect the foot resulting in exudating wounds. These ulcers may be on the bottom or under sole of the foot, on the toes, between the toes, on the top of the foot, on the arch of the foot and even on the ankles and heels of the foot. Diabetics may have more foot ulcers sometimes related to a complication called neuropathy that causes the individual to lose feeling in his/her/their feet. A scrape, cut or puncture in the skin can turn into an ulcer, but the individual might not know it's there if they have neuropathy and do not feel the wound or pain from the wound. Ulcers can lead to infections and in extreme cases the infection will not heal resulting in partial amputation of the individual's foot or toe(s). It is estimated that about 15% of people with diabetes in the United States will get a foot or toe ulcer and around 14% to 24% of people with diabetes in the U.S. need an amputation after they get an ulcer. Diabetic foot ulcers are the leading cause of foot amputations. As of 2020, of the estimated 537 million people worldwide who had diabetes about 19% to 34% will develop a diabetic foot ulcer in their lifetime. Unfortunately, of those individuals who develop a diabetic foot ulcer, approximately 20% of them will require lower-extremity amputation, either minor (below the ankle), major (above the ankle), or both. More than 80 percent of amputations start with a foot ulcer. Even more extreme, of those who develop a diabetic foot ulcer about 10% will die within one year of their first diabetic foot ulcer diagnosis. Further, foot and toe ulcers can happen to many people but might be more common in Black, Native American and Hispanic people. If the individual has an eye, kidney or heart disease related to diabetes, they are also at a higher risk. Of those about 15% of people with diabetes with an ulcer, the ulcer is typically on the bottom of their foot. Some of those people will be hospitalized because of complications.

Individuals are also at a higher risk of getting foot and toe ulcers if they have any of the following conditions: blood circulation issues, heart disease, obesity, a foot condition like a bunion or hammertoe, kidney disease, and some lifestyle behaviors of using tobacco and alcohol. Ulcers are open wounds in the skin that can take on almost any shape. Some shapes are more common than others on specific parts of the body. For example, ulcers on the foot or toe might be shaped like a crater or a wedge. The ulcer can go down even into the bone resulting in amputation or even death.

Neurotrophic ulcers occur primarily in people with diabetes, although they can affect anyone who has impaired sensation in their feet. They can be found anywhere on the feet but typically develop on the parts of the feet and toes that are most sensitive to weight (pressure points). Neurotrophic or diabetic ulcers on the foot can occur on the sole of the foot, heel, and under the toes as well as the top of the arch. While neurotrophic or diabetic ulcers don't cause pain based on the lack of feeling on the foot due to neuropathy, this can be worse because the individual will not realize they have an ulcer until it is weeping or exudating. Further, autonomic neuropathy is associated with decreased production of sweat and components of the natural moisturizing factor leaving the skin dry that leads to disruption of the epidermal barrier resulting in dryness and the presence of fissures or cracks that may become a portal of entry for bacteria on the foot and even in between the toes. Areas with blisters, cuts, scratches, redness, or hemorrhage are indicative of local damage from local trauma, friction, or shear commonly caused by walking barefoot and poorly fitting footwear. Callus formation is a reaction to increased local pressure and heralds impending skin breakdown and ulceration. Yet another complication is a fungal infection which is not uncommon in the feet of diabetics. The most occlusive areas are the fourth and fifth toe web spaces allowing excessive moisture accumulation that promotes fungal proliferation as evident by skin maceration in the area as yet another complication on diabetic foot. Diabetic foot ulcers that aren't treated promptly can cause major complications. In addition to being significantly more susceptible to ulceration, a diabetic patient will also generally take a significantly longer period for such ulceration to heal. It is not uncommon for it to take 10-12 weeks for an ulcer on the foot of a diabetic patient to heal even when using a cast or other more permanent treatment or therapy where the individual may be on crutches with complete off-loading of any weight or pressure on the foot. In comparison, such an ulcer would likely heal in less than seven days in a healthy individual. A need exists for a wound dressing that is easy to apply by the individual alone or by a medical professional, exudates the ulcer or wound, improves healing times, reduces complications and assist in healing overall.

Arterial ulcers may be caused by a condition called peripheral arterial disease which reduces blood flow to your extremities. When this happens the foot tissue may start to die. The ulcers that form from reduced blood flow are called arterial ulcers. Arteries are blood vessels that transport blood from the heart to the rest of the body, including the feet and toes. Anyone can get an arterial ulcer, but individuals who smoke or have diabetes, high blood pressure or high cholesterol are at higher risk. Unlike neurotrophic ulcers, arterial ulcers can form on many parts of the foot, including on the tips of the toes, between the toes typically where the toes rub together, the nail bed on the top of the toes, and the bony parts of the feet and toes that rub against bed sheets, socks or shoes, and even.

Both diabetic ulcers and arterial ulcers are wounds or open sores that do not heal, or otherwise recur repeatedly, and cause persistent swelling as well as burning, itching, irritation and discoloration of the skin. Conventional treatment and therapy for such ulcers generally includes topical protection of the wound, as well as compression and antimicrobial treatment of the affected area. Other treatment plans include draining the ulcer or wound site, orthotics to try to reduce or shift pressure points on the foot, removing pressure from the area by wearing a cast, elevation of the foot for extended periods of time (which is difficult for individuals working), debridement and removal of infected tissue, plantar exostectomy which is removal of parts of the sole of the foot, shaving or removing bones that in infected, tenotomy which is removal of scar tissue that has developed at the ulcer or wound site, and even reconstructive surgery using skin grafts to replace the infected ulcer or would site.

Other wounds that need immediate attention may be emergency situations such as automobile crashes, explosions, fires, and other traumas. The medical professionals in that instance may be an Emergency Medical Technician in an ambulance arriving at the scene of a trauma and needing to control bleeding immediately. Time is of the essence in such situations and a pre-formed absorbent wound dressing would greatly enhance bleeding control and reduce risk of contamination at the wound site. For example, head wounds bleed at a higher rate than leg wounds due to a large amount of tiny arteries and veins serving the individual muscles and skin on the head, including superficial arteries and veins close to the surface of the skin on the head. It is noted that sometimes even small lacerations to the head can lead to very large amounts of bleeding that need to be controlled immediately. Thus a need exists for a pre-formed wound dressing for application to the scalp and/or head.

There are a number of tapes and bandages that are available for application to a wound site or ulcer but the anatomy and structure of the foot, ankle, leg, knee, though, hip, shoulder, arm, elbow, wrist, hand, neck and head causes problems with adhesion, pressure complicating any treatment and healing, as well as leakage or exudating of the ulcer onto the individual's clothing, bedsheets, couch and the like. A need exists for a wound dressing on the foot, ankle, leg, knee, though, hip, shoulder, arm, elbow, wrist, hand, neck and head for ulcers or any type of wound. Notably, common wounds, scrapes, cuts and the like on the foot, ankle, leg, knee, though, hip, shoulder, arm, elbow, wrist, hand, neck and head take longer to heal even in healthy individuals again due to the anatomy and structure of the target body part. Further complicating the situation may be traumas such as surgery, car accidents, explosions, fires, and other emergencies where there is massive bleeding and time is of the essence in order to stop the bleeding and triage the wound site whether on a bony prominence or certain anatomy as well as any other treated body part.

A need exists for a wound dressing for any wound on the foot regardless of whether the individual has diabetes, circulation issues or is a healthy individual with a foot wound. As of 2015, costs related to diabetic foot ulcer s alone were greater than $1 billion annually and rising yearly. As of 2022 it was estimated that overall diabetes care in the U.S. accounts for an estimated $273 billion in direct and $90 billion in indirect costs annually. Foot complications such as ulcers and infections represent a major source of costs among people with diabetes, leading to higher rates of hospital admission, emergency department visits, outpatient visits and home health care utilization, and excess annual expenditures of 50% to 200% above the baseline cost of diabetes-related care. Advanced-stage ulcers cost upwards of $50,000 per wound episode, and direct costs of major amputation are even higher. These numbers likely underestimate the true economic burden of diabetic foot ulcers given out-of-pocket expenses, loss of productivity, and decreased employment associated with diabetic foot ulcers. A need exists for a treatment plan to improve healing times and outcomes while reducing complications such as infections and amputations for diabetic foot ulcers as well as arterial ulcers, while also improving function and quality of life and reducing health care costs.

For those individuals with circulation issues, foot and leg ulcers are problematic as the muscles, veins and arteries are not healthy in the individual's leg or foot resulting in worsening ulcers. Normally, a healthy muscle, for example, a healthy leg muscle squeezes the deep veins of the legs and feet to help move blood back to the heart. One-way valves in the deep leg veins keep blood flowing back towards the heart. However, prolonged periods of standing or sitting can cause the walls of the deep leg veins to stretch. Over time, in susceptible individuals, this can weaken the vein walls and damage the valves, causing blood to pool in the veins and increase venous blood pressure. This may result in a condition known as chronic venous insufficiency (CVI). Treatment of CVI typically involves the use of compression stockings or medical hosiery to decrease chronic swelling. Compression stockings are elastic stockings that squeeze the veins to improve venous circulation and prevent excess blood from flowing backward. Compression stockings can also help to heal skin sores or stasis ulcers that often present in conjunction with CVI. It is also common to employ compression bandages to apply pressure to the leg. In this regard, a bandage is applied with constant tension so as to produce graduated compression with the highest pressure at the ankle. However, the technique is difficult and is often done by highly skilled healthcare professionals.

The current method of treatment for ulcers, typically on the lower leg, foot, thigh, hip, torso, shoulder, arm, elbow, wrist, hand, neck and head includes placing a wound dressing over the ulcer or skin area to keep the skin moist and prevent infection from entering the open wound or sore and to assist in healing or closing the open wound or ulcer. Depending on the patient though this time frame can be much longer than days but lead into weeks or even months where the ulcer does not close or heal and in some patients the ulcer never fully heals or closes. Currently venous and related wound dressings are limited to various bandages and dressings that the care giver must wrap around the lower leg and then add some sort of securing means such as surgical tape or other medical grade tape. The care giver must learn how to wrap the dressing around the target ulcer skin area to keep it tight so that the open sore does not ooze onto the patient's clothes or bed linens while not wrapping it too tight to inadvertently cut off circulation in the limb. This is a very long learning curve in managing the tightness at the right level for the caregiver to learn over time. The actual patient usually cannot apply the wound dressing themselves as they may be bed ridden or non-ambulatory. Thus, a need exists for an absorbent wound dressing that is pre-formed in the shape of a limb or extremity which can be used with a compression wrap in the treatment of venous ulcers, CVI, lymphedema and DVT.

Further, the state of the art of bandages has been the same for centuries, using cotton and gauze material to absorb the exudate such as blood, pus, weeping edema and the like. Historically, wet-to-dry dressings have been used extensively for wounds requiring debridement. In 1600 BC, it is noted that linen strips soaked in oil or grease covered with plasters was used to occlude wounds. Traditional wound dressing products including gauze, lint, plasters, bandages (natural or synthetic) and cotton wool are dry and used as primary or secondary dressings for protecting the wound from contamination. Gauze dressings made out of woven and non-woven fibers of cotton, rayon, polyesters afford some sort of protection against bacterial infection. Some sterile gauze pads are used for absorbing exudates and fluid in an open wound with the help of fibers in these dressings. These dressings require frequent changing to protect from maceration of healthy tissues and thus are costly and time consuming, both for the patient and the medical profession. Further, due to excessive wound drainage, dressings become moistened and tend to become adherent to the wound making it painful when removing.

Generally in the 1980s-1990s wound dressings developed to facilitate the function of the wound rather than just to cover it. These dressings are focused to keep the wound from dehydration and promote healing. Current conventional wound dressings may be based on synthetic polymers and are classified as passive, interactive and bioactive products. Passive products are non-occlusive, such as gauze and tulle dressings, used to cover the wound to restore its function underneath. Interactive dressings are semi-occlusive or occlusive, available in the forms of films, foam, hydrogel and hydrocolloids. These dressings act as a barrier against penetration of bacteria to the wound environment. Current wound dressings are supposed to provide moisture while also absorbing fluids and include hydrogels, hydrocolloids, alginates, synthetic foam dressings, silicone meshes, tissue adhesives, vapor-permeable adhesive films and silver/collagen containing dressing. However, the need to combine the wound dressing with compression therapy remains problematic for both the user and the medical profession. The level of compression and the maintenance of that compression level needs to be addressed while at the same time a wound dressing underneath the compression garment has to exudate the wound but still maintain some level of air.

Historically for the last century or longer bandages were the main wound dressing. There are three main components to typical wound dressing, for venous ulcer wounds or lymphatic wounds or wounds related to edema and leg swelling. Bandages was key role to do multiple steps in the manage of wound care. Sometime the bandage acted as a wound dressing, absorbing the drainage aka exudate management while also providing very limited compression therapy. Bandages for the last 30 years included wound dressing technology adding optional materials (alginates, foams, skin substitutes) and properties (silver impregnated). Bandages have progressed by adding multiple layers and improving the compression properties. Multi-bandage can absorb more fluid and have slower compression decay with more inelastic properties than a single layer gauze bandage. But the problem remains that the bandage, even if multiple layer, needs separate layers to manage the wound exit exudate and a then add a second or multiple layers for compression. This treatment plan requires a skilled care giver to do it properly and very importantly such compression bandages are not reusable. In use, such conventional and known compression bandages must be cut off to see the wound below the wrapper bandage. This in turn traps the patient into being seen by a clinician if there is treatment and severely limits the patients ability to engage in common tasks such as bathing, etc. Such a need exists for an absorbent bandage in combination with a compression garment that can be removed by the patient without medical assistance and is reusable, both to save cost and time on behalf of the patient and all medical professionals such a clinicians.

Another problem exists especially with exudating or draining wounds in that any compression bandage once becoming wet in contact with the exudating liquid, loses most if not all compression levels. Further, the liquid is trapped against the skin of the patient causing discomfort, irritation, maceration or worsening of the symptoms, infection and other complications. Venous ulcers that exudate have metabolic waste and proteins that prevent healing so leaving the fluid in contact with the wound is counter-productive for healing. Having a waterproof backing layer is not sufficient as the wound still requires some level of measurable air to assist in healing. Thus a need exists for a wound dressing compression system that has measurable air while not promoting leakage onto the patient's clothes or bedsheets or furniture, exudates the wound, applies compression therapy at a consistent level and is reusable.

From a lifestyle and economics view, most visits to medical professionals such as clinicians are for venous wound care are for everyday dressing changes and not for monitoring the progress of the wound, thereby causing increased costs (to the patient, the insurance company and even loss of other billable time for the medical provider) and time management of all involved. This creates a large burden to health care system due to medical professionals such as clinicians basically removing and reapplying compression bandages every few days for patients resulting in lost opportunity to do other procedures and cost to the insurance companies, Medicare, and the patient's co-pays every few days for weeks, months or even years on end. For the medical professional who is tasked with removing and then applying a new compression bandage, he or she will cut off and remove the typically soaked foul smelling bandage with debris flying around and leaking liquids, pus, blood and the like, then cleaning off the exudating and foul-smelling wound site and finally having to wrap the new replacement compression bandage around and around the wound site. This is a messy, odorous and arduous procedure which the clinical or medical professional may repeat ever few days for every single patient, over and over again for weeks, months or even years. Further, the patient has multiple burdens on his or her time to travel to and from the medical office every few days, economic costs and notably reduced quality of life as not able to engage in everyday activities such as bathing and the like. In addition, the clinical or medical professional may need an assistant depending on the body part being treated as in certain cases a swollen leg can weigh 100 pounds itself, which needs to be lifted while being conventionally bandaged. A need exists for a pre-formed wound dressing that the user himself/herself can apply with their own two hands, or a clinical or medical professional can apply without needing assistance from a second clinical or medical professional.

The treatment using conventional compression bandages may not be effective for certain conditions such as lymphorrhea where the entire leg weeps resulting in drainage through the skin without a specific ulcer being present on the skin. Further, for some post-operative patients there is localized swelling or acute swelling or seepage from a surgery cut resulting in high level of exudate or liquid to be absorbed by the compression bandage. Again, in these instances the compression level of the compression bandages is significantly reduced or even eviscerated to zero compression. Thus a need exists for a wound care compression system that can compress the limb while absorbing exudate and fluids such as blood or pus while at the same time maintaining the target compression level. Indeed another problem with maintaining the compression level of a compression bandage is that once the immediate swelling is reduced then the compression level is directly reduced as the circumference of the limb is lower resulting in gaps and lower compression or zero compression.

Another conventional wound dressing of plasters or other such as the Unna boot may be helpful for the impregnated additives to assist healing, but such materials and wound dressings do not have compression. Such materials may be comprised of a pad or alternative wound contact dressing with impregnated materials but no compression therapy is available even when first applied, never mind when the initial swelling is reduced and then there is a gap between the skin of the patient and the set plaster or Unna boot.

The inventive pre-formed wound dressing and system solves these and other problems and fill many needs by providing a multi-layer system which is comfortable, non-stick, absorbent, and has backing layer that prevents strike through leakage onto the user's clothes or other materials such as furniture while at the same time enabling vapor transfer so it is not occlusive which no build up in Co2 or bacteria growth, thus assisting in healing and pain relief while reducing complications to the patient and also providing cost savings to the user and the medical field as a whole. The inventive pre-formed wound dressing and system addresses these and other problems and issues for the medical field and the patient or user. A need for a wound dressing compression system that provides measurable air to the wound site, a compression level that is maintainable throughout the treatment plan, is capable of self-application by the patient including being able to remove the system and reuse it, is met by the inventive wound dressing compression system. Notably, anyone can properly place the wound dressing compression system on the user in a more effective way than known conventional compression bandages, which reducing edema and assisting in wound healing.

As stated above, many known wound dressing and bandages have added waterproof backings to assist in leakage of exudate problems, but for compression needs the waterproof backing reduces the compression levels. This is notably important for treating venous ulcers, pressure ulcers, and conditions such as edema, lymphedema, chronic venous insufficiency (CVI), Deep Vein Thrombosis (DVT), Cerebral Venous Thrombosis (CVT), and other types of conditions related to circulation, veins, and pressure points as well as emergency traumas being treated at the location outside a medical facility such as an automobile accident, fire, explosion, gun shooting, war zone and the like. Turning to certain treatment plans or methods to treat CVI and lymphedema, compression bandaging is typically employed but has several disadvantages. The bandaging is time consuming, and the effectiveness is limited to the skill of the provider. In some instances, bandages can be applied too tightly or too loosely and may slip from their intended position, decreasing their effectiveness. When this occurs, bandages must be taken off and reapplied, further increasing the time of application and decreasing the consistency of application of the therapy. Further, when the compression bandaging is on the thigh area of the leg, the bandaging slips down the leg when the user walks or moves around even in bed, possibly due to the anatomical shape of the thigh tapering towards the knee area as well as down the calf toward the ankle when worn on the lower leg. Further, compression dressing is typically non-absorbent so therefore the patient has leakage or oozing from the wound such as a leg ulcer, that leaks onto bed sheets or clothes. Thus a need exists for a pre-formed absorbent wound dressing to apply to the patient or user's limb or extremity more easily than current wound dressings.

The effectiveness of many of the current compression therapies is also limited by the application of current products. Because current compression therapy is done either with manual wraps or electromechanical systems, they require either a skilled medical professional to apply and/or the need for the patient to be stationary for extended periods of time. Although stockings and/or bandages can be worn by patients and self-administered, they are very difficult for the patient to put on and pose a challenge for unskilled medical professionals to apply consistently and effectively. Again though, the stockings and/or bandages will slip down the thigh towards the knee or down the calf to the ankle once the user walks or moves around. The same is true for bandages or wound dressing on the lower or upper arm, torso, chest, neck or head as well.

CVI and lymphedema may also result in DVT, which is an affliction that causes blood clots particularly in the lower extremities of the legs. When a patient is not ambulatory, the patient faces an elevated risk factor of creating a blood clot. These blood clots, which often accumulate or reside in the patient's calf or thigh, are not, in and of themselves, overly dangerous. However, when the blood clot breaks loose, they create a pulmonary embolism which can get lodged in the patient's heart, brain or lungs where it can cause significant damage or death. It is estimated that in each year 2 to 2.5 million Americans are afflicted by DVT causing 600,000 patients to seek medical care with 300,000 patients succumbing to the effects of the pulmonary embolism. Among people who have had a DVT, one-third will have long-term complications (post-thrombotic syndrome) such as swelling, pain, discoloration, and scaling in the affected limb. Further, about one-third (about 33 percent) of people who are diagnosed with blood clots will have a recurrence within ten years. Thus, a system and apparatus and method of use to prevent, reduce and/or treat DVT is needed which is easy to use by the patient at home, ambulatory in that the patient can work and walk about both inside and outside of the home, and less cumbersome than current known DVT systems. The number of Americans who suffer from DVT may be affected or have an increased risk based on (a) recent surgery, which decreases a patient's mobility and increases inflammation in the body, which can lead to clotting; (b) medical conditions that limit mobility, such as an injury or stroke; (c) long periods of travel, which limit mobility; (d) injury to a deep vein; (e) inherited blood disorders that increase clotting; (f) pregnancy; (g) cancer treatment; (h) smoking; (i) obesity; and (j) many other conditions. Thus, a system and apparatus and method of use to treat DVT is needed.

Many of the current treatment options for CVI and lymphedema cause venous ulcers including the use of current known devices, apparatus, bandages, stocking, hosiery and the like. A venous ulcer is damage and loss of skin above the ankle that is the result of a problem with the veins in the leg. Venous ulcers typically develop on either side of the lower leg, above the ankle and below the calf. They are difficult to heal and often recur. They also develop on the thigh, on the inner portion of the leg thigh or outer thigh area as well as near the groin area on the leg. Further, venous ulcers may develop around the knee, behind the knee, along the sides and on the front of the knee as well. These venous ulcers may ooze blood, pus or other liquids thus requiring constant wound dressings applied to the target area or limb. Current wound dressings lack support or shape of the target limb or extremity and are instead typically flat and wrapped around and around the target limb or extremity. A need exists for a pre-formed absorbent wound dressing in the shape of the target limb or extremity.

The veins of the leg are divided into the superficial and deep systems according to their position relative to the fascia. The deep veins, which come together to form the popliteal and femoral veins lie within the fascia and are responsible for the venous return from the leg muscles. Dilated valveless sinusoids also lie within the fascia (more particularly in the soleus and gastrocnemius muscles). The sinusoids fill with blood when the leg is at rest.

The long saphenous vein which runs along the medial side of the leg from foot to groin and the short saphenous vein which runs at the back of the calf from foot to knee are the major vessels of the superficial venous system. These vessels lie outside the fascia and are responsible for the venous return from the skin and subcutaneous fat. Compression on the long saphenous vein may be recommended in treatment of certain conditions whereas in other conditions there may be less compression recommendation on the long saphenous vein on the medial side of the leg and more compression recommended on the short saphenous vein of the leg which runs on the back portion of the leg, including the thigh. Communicating veins, sometimes called perforators because they perforate the deep fascia, join the two systems. The perforators, like the other veins in the leg, contain valves that permit the flow of blood in one direction only, from the outer or superficial system inwards to the deep veins.

The venous pressure at the ankle of a subject who is lying supine is around 10 mmHg, but on standing this will rise considerably due to an increase in hydrostatic pressure (equivalent to the weight of a vertical column of blood stretching from the point of measurement to the right auricle of the heart). During walking, as the foot is dorsally flexed, the contraction of the calf muscle compresses the deep veins and soleal sinuses thereby emptying them of blood. As the foot is plantarly flexed, the pressure in the veins falls, the proximal valves close, and the veins are refilled by blood passing through the perforators from the superficial system. During this cycle, in a normal leg, the distal valves of the dee veins and the valves of the perforators will ensure that the expelled blood can go in only one direction—upwards, back to the heart.

Blockage or damage to the venous system will cause disruption to normal blood flow, which may manifest itself in a number of different ways according to the site and extent of the damage. If the valves in the superficial system are affected, venous return will be impaired and blood may accumulate in the veins causing them to become distended, leading to the formation of varicosities (varicose veins). Such varicosities may be located in the thigh, knee, calf, ankle or foot area of the user's leg.

If the function of the perforator valves is impaired, the action of the calf muscle pump will tend to cause blood to flow in the reverse direction into the superficial system increasing the possibility of damage to the superficial vessels. Again, the current compression therapy is done either with manual wraps or electromechanical systems, they require either a skilled medical professional to apply and/or the need for the patient to be stationary for extended periods of time. A need exists for a new system to treat such medical issues. There may be minimal blood flow upward into the thigh and the rest of the body, causing issues for the patient along the body including the thigh, calf, ankle and entire leg of the patient.

Following a deep vein thrombosis that results in complete or partial obstruction of a deep vein, the unrelieved pressures produced by the calf muscle pump on the perforator valves may cause these to become incompetent. If this occurs, there will be a large rise in the pressure in the superficial system, which may force proteins and red cells out of the capillaries and into the surrounding tissue. Here, the red cells break down releasing a red pigment that causes staining of the skin, an early indicator of possible ulcer formation. The ulcer formation can be in any part of the body, including the leg, groin, thigh, knee, calf, ankle and foot.

Venous leg ulcers are generally shallow and red in color. The skin surrounding the ulcer is frequently discolored due to the staining described previously. Incompetent perforating vein valves can also cause malleolar venules to become dilated and appear as fine red threads around the ankle as well as the thigh, knee, calf or foot are of the leg or any area of the leg. This condition, called ankle flair, is also diagnostic of a venous ulcer. The condition may also be seen on the thigh, including the inner medial thigh area or outer lateral thigh area, or around the knee or groin area of the patient's body.

Arteries transport oxygen replenished blood from the heart to the rest of the body. Veins return oxygen depleted blood back to the heart. When the veins in the lower extremities of the body have difficulty transporting blood back to the heart, a condition develops called chronic venous insufficiency (CVI), also known as chronic venous disease (CVD). CVI. Most commonly occurs as the result of a blood clot in the deep veins of the legs, a disease known as deep vein thrombosis (DVT). CVI also results from pelvic tumors and vascular malformations, and sometimes occurs for unknown reasons. When a person is standing or sitting, blood in the veins of the legs flows in an upward direction. When the person walks, the calf muscles and muscles in the feet contract to squeeze the veins and push the blood upward. To keep the blood flowing upward and prevent it from flowing downward, the veins contain one-way valves. CVI occurs when these valves become damaged and allow the blood to leak back downward in the opposite direction. Such valve damage may occur as the result of aging, extended sitting or standing, or a combination of aging and reduced mobility. When the veins and valves become weakened and the blood does not properly flow up to the heart, blood pressure in the veins of the lower extremities can stay elevated for long periods of time, leading to CVI. This condition is more common in older individuals, and if not properly treated, can lead to burst capillaries, local tissue inflammation, internal tissue damage, varicose veins, ulcers, and open sores on the skin's surface. The burst capillaries can be seen on the patient's leg including the thigh, knee and groin.

CVI can diminish the capacity of the venous system and increase the workload of the lymphatic system in the affected area. The lymphatic system must then transport larger volumes of water and protein to reduce the fluid load in the affected tissues of the legs, a situation which is especially difficult for patients with lymphedema, varicose veins, and other lower extremity pathology. One non-surgical option often used to help prevent or treat the leg extremity pathologies discussed above is the use of compression stockings. Compression stockings help prevent leg fatigue, ankle and foot swelling, spider veins, and varicose veins. They improve circulation in the legs, especially when used in conjunction with frequent exercise and leg elevation. Compression stockings maintain pressure on the legs while allowing for normal ambulation. Increasing pressure in the tissues beneath the skin reduces excess leakage of fluid from the capillaries and increases absorption of tissue fluid by the capillaries and lymphatic vessels. In addition, the increased pressure decreases the size of the veins, which causes the blood to flow faster and helps prevent it from pooling.

Compression stocking tightness typically varies between 15-50 mm HG. The tightness of a given stocking depends on its particular configuration- and class. For example, stockings having a compression pressure of 15-20 mm HG are considered light compression stockings. Class I stockings are 20-30 mm Hg, class II stockings are 30-40 mm Hg, and Class II stockings are 40-50 mm Hg. While such compression stockings are a commonly utilized non-invasive treatment of leg pathology, the issues they present are numerous. Wearing a tightly fitting stocking can be tedious or time consuming to put on, and may require help from another person if the wearer is injured, elderly, or has some form of disability. Any tight-fitting stocking to be worn on the thigh area has a harder time being put on as the user has to pull the tight-fitting stocking over the foot, ankle, calf, knee and finally up to the thigh area. Again, due to the shape of the thigh, these tight-fitting stockings will slip down the thigh at some point due to the user walking or moving about. In addition, the pressure applied by the stocking generally stays relatively constant during use without any option of increasing or decreasing the tightness level. As compression stockings are repeatedly worn, they lose elasticity and thus tightness over time. Once such prescribed elasticity and tightness is lost, the stocking is of little or no value, and needs to be replaced on account of its looseness, which requires buying a new pair to obtain the desired pressure. Further, given the shape of the thigh, the top or upper portion of the stocking must be very tight in order to prevent slippage down the thigh, which can in turn lead to complications of its own due to the excessive tightness. Typical compression stockings are also non-absorbent so require an additional wound dressing on the target area of the ulcer to be applied and then the compression stocking is pulled up over the target limb or extremity. A need exists for a pre-formed absorbent wound dressing in the shape of the target limb or extremity to be applied or placed over the ulcer either alone or as part of a wound dressing compression system.

Medical hosiery represents a useful and convenient method of applying compression to normal shaped legs in order to prevent the development or recurrence of leg ulcers. However, these stockings are of limited value in the treatment of active ulceration, being difficult to apply over dressings. In such situations compression bandages currently represent the treatment of choice. Compression bandages apply a pressure to the limb that is directly proportional to bandage tension but inversely proportional to the radius of curvature of the limb to which it is applied. This means, therefore, that a bandage applied with constant tension to a limb of normal proportions will automatically produce graduated compression with the highest pressure at the knee. This pressure will gradually reduce up the thigh as the circumference increases.

As can be readily appreciated, it is cumbersome and difficult to apply uniform tension to the compression bandage as it is applied to the treated limb, and thus this is accomplished only by highly skilled caregivers. Moreover, once secured to the treated limb, care and attention must be given to ensure that the bandage does not slip or become displaced as this will lead to multiple layers forming, which in turn may lead to localized areas of high pressure, which can place the patient in direct risk of skin necrosis. Further, as most compression dressings are non-absorbent, the provider must apply two layers of dressing, first an absorbent wound dressing and then a compression dressing, requiring even more skill for both dressing applications.

DVT is widely recognized as a major risk factor facing patients who undergo total hip arthroplasty (THA) and total knee arthroplasty (TKA). Without prophylaxis (preventive treatment), up to 80 percent of orthopedic surgical patients will develop DVT, and 10 to 20 percent will develop PE. Even when proper prevention measures are taken, it is estimated that 3 percent of orthopedic surgical patients will develop DVT, and 1.5 percent will develop PE. DVT and PE remain the most common cause for emergency re-admission and death following joint replacement surgeries. In one survey conducted the patients surveyed after a THA or TKA stated problems with their prophylaxis varied widely: 83 percent reported issues with lack of ambulation, 74 percent used compression stockings, 57 percent used mechanical compression, 58 percent used an anticoagulant pill, 46 percent used an anticoagulant injection, and 42 percent used aspirin. (https://www.stoptheclot.orn/about-clots/toolkir-for-knee-hip-replacement-patients/orthopedic-surgery-fact-sheet/). Thus, a need exists for a prophylaxis which is easy for the patient to use at home or work (outside a hospital setting or with the aid of a medically trained professional as noted above) which is ambulatory so that the patient can walk and return to life activities.

Knee replacements may also result in DVT as a post-operative complication. After knee surgery, most DVTs occur in the calf. Although less likely to lead to a PE, these clots are more difficult to detect. Less than one third of patients with DVT present with the classic signs of calf discomfort, edema, distended veins, or foot pain. It has been noted that the risk of developing DVT extends for at least three months after a total knee replacement. The risk is greatest two to five days after surgery; and a second peak development period occurs about 10 days after surgery. Currently, it has been noted that patients at home are experiencing an increase in DVT due to lack of activity and movement. While the patient is in the hospital, he or she may be connected to an electrical powered pump in combination with a therapeutic compression apparatus, however, once discharged, the current products are limited as noted herein, whereby most therapeutic compression apparatus are manual pumped so the patient can walk, work, etc. and those that are electric powered typically require being tethered to an electric outlet or the electric pump is integral to the therapeutic compression apparatus and not practical to walk about in life. Thus a need exists for a system which can be used to prevent, reduce or even treat DVT which is practical to use, mobile and easy to be administered by the patient post-operative total knee replacement of any other knee, hip or leg surgery. A need also exists for a pre-formed absorbent wound dressing applied over a surgical wound that is healing which can be opened to check on the wound site and closed again with one or two hands without the need for a medical professional or skilled caregiver. Further, a need exists so that the user can walk around with the compression profile maintained while not being limited to an electrical plug or some other separate source of continued, maintained or even increased then decreased compression.

Further compression treatments have been discussed in “Effect of High-pressure, Intermittent Pneumatic Compression for the Treatment of Peripheral Arterial Disease and Critical Limb Ischemia in Patients Without a Surgical Option”, by Oscar M. Alvarez, Martine E. Wendelken, Lee Markoqitz and Christopher Comfort (Wounds, Vol. 27, Issue 11, pages 293-301, November 2015) wherein thirty-six patients with symptomatic peripheral arterial disease (PAD) or critical limb ischemia (CLI) who were experiencing claudication pain, chronic resting pain, numbness, and ischemic lower leg/foot ulceration were randomized into 2 treatment groups. Eighteen of these patients received treatment with high-pressure, intermittent pneumatic compression (HPIPC) 60 minutes twice daily for 16 weeks, and 16 subjects received standard care consisting of an exercise regimen of walking for 20 minutes twice daily for 16 weeks. The HPIPC device delivers bilateral pressures of 120 mm Hg. Cycle times provide sequential compression for 4 seconds (+/−0.5 seconds) followed by a 16-second rest period (+/−3.0 seconds), resulting in a 20-second cycle or 3 cycles per minute. The study was designed to measure patient-centered outcomes. The primary endpoint was peak walking time (PWT), defined as time to maximally tolerated claudication pain. The conclusion at the end of the study was that therapy consisting of HPIPC for 2 hours daily for a period of 16 weeks significantly improved PWT, reduced resting pain, and improved healing rates, physical function, and bodily pain. There were no device related complications, allowing for long-term use. A further conclusion was that HPIPC offers an excellent alternative for the palliative care of patients with PAD and CLI symptoms. Thus, a need exists for a system which can be easily administered by a patient in the home setting which includes HPIPC.

Co-owned U.S. Publication No. 2004/0193084, which is hereby incorporated by reference herein in its entirety, discloses a device for applying pressure to the human leg for use in conjunction with treatment of varicose veins. The device includes a flexible member and at least one air bladder chamber integral thereto that are adapted to securely wrap around the human leg. A tube in fluid communication with the air bladder chamber(s) extends to an air pumping mechanism that operates to inflate the air bladder chamber(s) to a pressurized state. The flexible member preferably includes an opening at the knee joint level to enable a patella to protrude therethrough. In addition, the flexible member preferably extends below knee joint level and is adapted to securely wrap around a lower portion of a leg to provide stability to the leg. Preferably, the air bladder chamber of the device is substantially longer in a first dimension than in a second dimension orthogonal thereto such that the air bladder chamber can be positioned to cover a portion of the human leg that is relatively long in the vertical dimension and narrow in the horizontal dimension.

Co-owned U.S. Pat. No. 7,276,037, which is hereby incorporated by reference herein in its entirety, discloses an apparatus for applying compression therapy to an extremity of the human body, such as a portion of the human leg. The device includes a flexible member and an air bladder chamber. The flexible member is adapted to wrap around the extremity to secure the air bladder chamber to the extremity. An air pumping mechanism is operated to inflate the air bladder chamber to a pressurized state. One or more fluid-filled pressurized members are provided, each separate and distinct from the flexible member and the air bladder chamber and thus readily moveable relative to the flexible member and the air bladder chamber. The pressurized member(s) is operably disposed between the extremity and the flexible member whereby it applies increased localized pressure to the extremity during use. Preferably, the air bladder chamber is substantially longer in a first dimension than in a second dimension orthogonal thereto such that it can extend longitudinally along the extremity to cover a relatively long and narrow portion of the extremity. The position of the air chamber can be readily adapted to apply local pressure to desired body parts (such as a certain venous channel). The pressurized member(s) can be positioned during use such that it covers a venous ulcer (or other treatment sites) and applies increased localized pressure to the treatment site in order to promote healing.

A need exists for a pre-formed absorbent wound dressing in the shape of a selected or target limb or extremity. Such a pre-formed absorbent wound dressing is easier to place on the wound site, ulcer or surgical site wound and lasts longer than common bandages and wound dressings. The pre-formed absorbent wound dressing is part of a wound dressing compression system including a therapeutic compression apparatus having a sufficient SSI or greater than 10-12 applied over the pre-formed absorbent wound dressing as current known apparatus, devices, bandages, stockings and hosiery, though especially the current treatment inelastic apparatus and devices are only capable of a SSI of no higher than about 10-12 so that a need exists for a therapeutic compression apparatus having a SSI of above about 12, preferably about 15-25. Other uses for the inventive pre-formed absorbent wound dressing and related compression system may be envisioned.

### SUMMARY OF THE INVENTION

The subject invention is directed to a pre-formed absorbent wound dressing shaped for the target limb or extremity such as a foot, ankle, calf, thigh, knee, leg, torso, chest, shoulder, arm, wrist, hand, elbow, neck, head, scalp or any other limb. The inventive pre-formed absorbent wound dressing is comprised of three layers including a wound contact layer, an absorbent layer and a waterproof vapor layer and the wound dressing is configured in a shape to conform to the target limb. The target limb can be any body part but may be a foot, lower leg, though, arm or head of a person, as well as other body parts where wounds are present and hard to shape over such bony area or otherwise shaped area such as the head or scalp. The pre-formed absorbent wound dressing may also include an indicator for when the exudate within the absorption layer has reached a certain capacity level and the inventive pre-formed absorbent wound dressing needs to be replaced. The pre-formed absorbent wound dressing is also part of an inventive wound dressing compression system and methods of use. The wound dressing compression system includes one or more compression apparatus each having at least one bladder and depending on target limb may have two, three or more bladders, all connected to one inflation means and each apparatus having a check valve in an inflation port on the apparatus capable if maintaining the pressure within the inflated bladder when the inflation means is disconnected to the apparatus. Each therapeutic compression apparatus has a SSI of at least about 12 or greater. The wound dressing compression system includes a therapeutic compression apparatus and an inflation means wherein the inflation means is less bulky than known inflation means. The inflation means includes at least two settings, a setting of constant inflation or pressure and a setting of varying or intermittent inflation or pressure. The inventive wound dressing compression system may include at least one sensor, such as a motion sensor, pressure sensor, blood pressure sensor, tonometer sensor, or other sensors to monitor use of the inventive system by the patient and/or medical professionals. In use, the pre-formed absorbent wound dressing is placed on the target limb or extremity, secured, and then the therapeutic compression apparatus is placed over the pre-formed absorbent wound dressing, the apparatus is secured and then inflated via the inflation means. The wound dressing compression system may also include a dressing which is impregnated with an additive such as medication, vitamins, anti-bacterial coating, silver or zinc oxide. The wound dressing compression system may also include a wound bolster or other elevating apparatus such as a pre-filled bladder in combination with an absorbent foam or other material adhered to the wound site.

The pre-formed absorbent wound dressing is in a shape pre-formed for use on a limb, leg, calf, thigh, hip, pelvis, knee, foot, ankle, torso, arm, neck or other body parts. The pre-formed wound dressing has an inner layer that is in contact with the wound site and surrounding skin area, a middle absorbent layer for the exudate (which absorbent layer may itself be comprised of multiple layers), and an outer layer that is non-absorbent but allows moisture vapor transfer from the wound site to facilitate and assist in treatment at the wound site as well as healing, and a shaping component so that the preformed wound dressing is in the shape of the target limb or extremity. The pre-formed absorbent wound dressing includes a securing or closing means as well as in some embodiments an adjusting means. The wound dressing compression system includes a therapeutic compression apparatus which such apparatus may further comprise at least one bladder operatively associated with the therapeutic compression apparatus for applying pressure to a treatment site on the limb such as a leg having a SSI of at least about 12 or above. The therapeutic compression apparatus includes a connecting means such as a loop and hook material in order to wrap the therapeutic compression apparatus around the limb, such as a leg, of the user. Alternatively, the at least one bladder may be integral with the connecting means or wrap and depending on the target limb may have at least two or at least three separate bladders within the apparatus. One or more connecting means may be operatively associated along the first and second peripheral edges of the therapeutic compression apparatus for securing it around the limb. The at least one bladder may be adapted and configured to form a predetermined gradient compression profile and/or gradient pressure profile when the at least one bladder is filled. The gradient compression profile and/or gradient pressure profile may be determined by the location of various spot welds on the bladder to create the gradient compression profile and/or gradient pressure profile and when inflated the inventive apparatus has a SSI or at least about 12 or greater. The gradient compression profile and/or gradient pressure profile instead may be determined by the inflation means connected or integral to the therapeutic compression apparatus. In another embodiment, the gradient compression profile and/or gradient pressure profile may be determined by pressure being created in one direction within the bladder by the inflation means and then exiting the bladder through an exhaust port or other exit means. The at least one bladder may be one of a wedge-shaped bladder, a cone-shaped bladder, a disk-shaped bladder or a rectangular-shaped bladder. The at least one bladder may also include a plurality of fluid chambers. The therapeutic compression apparatus, as part of the inventive system, may further comprise at least one means for adjusting pressure coupled to the at least one bladder for controlling an amount of pressure supplied to the treatment site. In an apparatus with more than one bladder, each bladder is connected to the same inflation means and the inflation means sequentially inflates each bladder, which could be the same or different pressure levels, and once disconnected a check valve within an inflation port on each separate bladder maintains the then current pressure level.

The therapeutic compression apparatus may include an inelastic portion and an elastic portion, such as on the upper portion of the at least one bladder when worn on a lower leg, such as around the upper calf area of the limb. The elastic portion may be located near the elbow, wrist or shoulder of a user when placed on the arm limb. The elastic portion may be located at the groin area of the user when placed on the thigh or lower torso or hip or buttocks area of the user. Other locations may be employed depending on the limb being subject to the wound dressing compression system on the user. The therapeutic compression apparatus for providing pressure to a limb including a bladder assembly. The pressure profile and/or compression profile may be a gradient pressure profile and/or a gradient compression profile. The bladder assembly when inflated has a SSI of at least about 12 or greater. The top portion of the bladder assembly is adjacent to an elastic portion which may provide a tighter fit to the user when the entire inventive therapeutic compression apparatus is worn on the user's target limb.

An inflation means for inflating the bladder such as the air pocket through at least one inflation port may be provided in the first wall of the bladder assembly. The inflation means may be detachable from the at least one inflation port. At least one pressure valve may be operatively associated with the inflation means for controlling an amount of pressure within the bladder and the air pocket within the bladder. The inflation port includes a check valve so as to maintain a given pressure within the bladder of the therapeutic compression apparatus. The inflation port may be universal in that it is configured to be capable of connecting to and accepting a plurality of inflation sources and inflation means such as a manual pump, mechanical pump, electrical pump, battery-operated pump, static pump, intermittent pump, pneumatic pump, negative pressure source and other variations. Where there are more than one bladder within the apparatus, each bladder is sequentially inflated, and may be sequentially deflated and repeated on and on, or the pressure may be maintained once the inflation means is disconnected to the inflation port located on each separate bladder. The bladder is connected to an inflation port including a valve configured so that when the valve is in the closed position the pressure profile and/or compression profile is maintained at the then current pressure and/or compression level. The valve is configured so that when it is in the open position the fluid, such as air, flows from the inflation means into the bladder and if not connected to an inflation means then the fluid escapes from the bladder and it is deflated. The valve may be self-sealing or it may be connected to a means to open and close it with for example a level, knob, screw or other opening and closing means.

A method of the invention includes the per-formed absorbent wound dressing being placed at the wound site on the target limb by the patient or user and being able to secure it on the target limb with two hands alone and without the aid of a skilled medical provider or caretaker. The user places the inventive pre-formed absorbent wound dressing on the target limb, removes the adhesive tabs on the closing or securing means and places the adhesive portion on the outer layer of the pre-formed absorbent wound dressing to close and secure it to the target limb. The pre-formed absorbent wound dressing adhesive tabs can be opened and closed a number of times for the user to check on the wound site. Optionally, the pre-formed absorbent wound dressing adhesive tabs include an end portion that does not have adhesive but can be easily gripped by the user in order to open and close the adhesive tabs to check on the wound site. The closing or securing or fastening means may also be a hook and loop connection or other known securing means. The inventive pre-formed absorbent wound dressing can be used over any wound such as a surgical site, a cut (whether small or gaping in an emergency situation), an ulcer, a burn or any type of wound. The compression system including a therapeutic treatment apparatus used to treat CVI, DVT and/or lymphedema by applying the pre-formed absorbent wound dressing over the target limb or extremity, closing or securing it, then applying the primary and secondary wraps around the pre-formed absorbent wound dressing, closing or securing it and then inserting an inflation means into an inflation port and inflating the bladders within the primary and secondary wraps and maintaining a certain pressure to treat the CVI, DVT and/or lymphedema and having a SSI of at least about 12 or greater when inflated. Optionally a wound dressing impregnated with silver or zinc oxide may be placed over the wound site alone or in combination with the wound bolster. Another embodiment of the present invention includes an assembly according to the invention includes a pressure mechanism having a flexible member for attachment to a limb and an air chamber which may be pumped up into a desired pressurized state, a separate relatively small pre-filled air bladder, an absorbent foam, sponge or dressing coupled to the pre-filled air bladder, and a suction conduit coupled to a source of negative pressure (suction) and in fluid communication with the absorbent foam, sponge or dressing. In a preferred embodiment, the pre-filled air bladder, the absorbent foam, sponge or dressing and the suction conduit are formed together as a unit. According to one aspect of the invention, the flexible member of the pressure mechanism is adapted to wrap around a leg or arm and over the pre-filled air bladder in order to secure the pre-filled air bladder and the foam, sponge or dressing to a wound or ulcer in the extremity. Thus, the flexible member is provided with some fixation structure such as a hook and loop closure mechanism. An air pumping mechanism is preferably coupled to the air chamber of the pressure mechanism in order to inflate the air chamber to a pressurized state. The air chamber of the pressure mechanism is preferably designed to apply pressure along a predefined area (e.g., the saphenous vein of a leg) as opposed to around an entire limb. According to another aspect of the invention, the suction conduit is located either between the pre-filled air bladder and the absorbent foam, sponge or dressing which is adhered to the small air bladder, or the pre-filled air bladder is formed as a donut with a central opening and the suction conduit extends through the central opening. By coupling the suction conduit to a source of negative pressure, exudate from the wound or ulcer is sucked through the foam, sponge or dressing into the suction conduit.

One of the methods of the invention include locating the pre-filled air bladder and foam, sponge or dressing over a wound or ulcer on a limb, placing the pre-formed absorbent wound dressing on the target limb or extremity over the pre-filled air bladder and foam, sponge or dressing, then wrapping the flexible member of the pressure mechanism around a limb with the air chamber located over the pre-formed absorbent wound dressing and fastening the pneumatic pressure mechanism in place with the fixation structure. When the apparatus is properly located and affixed to the limb, the air chamber is inflated, preferably to 30-40 mm Hg, thereby applying pressure to the limb and more specifically via the pre-filled air bladder to the wound. The suction apparatus is activated by turning on the source of negative pressure, and exudate from the wound or ulcer is pulled through the absorbent foam, sponge or dressing into the suction conduit.

Another embodiment of the present invention includes a system including an apparatus for applying intermittent pressure to a portion of the human body, such as an area of the human leg, which assists with the healing and treatment of various conditions such as venous ulcers or wounds by promoting blood flow into and out of the area and by increasing drainage. The apparatus may include a thigh bladder or a foot bladder and a leg bladder, each having inflatable chambers (at least one and possibly two or more bladders within each apparatus) that accommodate an entering fluid by inflating. The bladders are fluidly coupled by a fluid conduit, and each is preferably equipped with a means for locating it on a portion of the body. In a preferred embodiment, the thigh bladder is positioned between the pelvis or groin or hip area of the user and the knee of the user. As a person walks while wearing the apparatus, a portion of the thigh bladder deflates as the person's leg moves foot (heel) strikes the ground due to the external pressure placed on the foot bladder, thereby forcing fluid out of the foot bladder, through the fluid conduit, and into the leg bladder, which raises the pressure therein. As the person's foot rolls from heel to toe in the standard walking motion, the external pressure from the person's weight is removed from the foot bladder, resulting in the pressure of the leg bladder being higher than the pressure in the foot bladder. Fluid thus flows back through the fluid conduit and into the foot bladder, which then inflates again to its original state, such that the pressures of the foot bladder and leg bladder are equalized. This process repeats as a person walks, thereby creating a pumping or kneading force on the leg as the pressure in the leg bladder intermittently increases and decreases, thereby promoting blood flow, fluid drainage, treatment, and healing to various parts of the leg.

In another embodiment including a pre-formed absorbent wound dressing in the shape of thigh and/or lower leg depending on the location of the wound or ulcer, further including a thigh therapeutic compression apparatus and an integral or separate lower leg therapeutic compression apparatus including a foot bladder, the foot bladder is positioned over a second pre-formed absorbent wound dressing placed around the ankle and foot of the user such that the foot bladder is applied over the wound dressing on a bottom of a foot and the leg bladder is positioned on a lower portion of a leg. As a person walks while wearing the apparatus, a portion of the foot bladder deflates as the person's foot (heel) strikes the ground due to the external pressure placed on the foot bladder, thereby forcing fluid out of the foot bladder, through the fluid conduit, and into the leg bladder, which raises the pressure therein. As the person's foot rolls from heel to toe in the standard walking motion, the external pressure from the person's weight is removed from the foot bladder, resulting in the pressure of the leg bladder being higher than the pressure in the foot bladder. Fluid thus flows back through the fluid conduit and into the foot bladder, which then inflates again to its original state, such that the pressures of the foot bladder and leg bladder are equalized. This process repeats as a person walks, thereby creating a pumping or kneading force on the leg as the pressure in the leg bladder intermittently increases and decreases, thereby promoting blood flow, fluid drainage, treatment, and healing to various parts of the leg.

In one embodiment of the present invention, the wound dressing compression system includes an inflation means connected to a belt so the user can wear it around his or her waist in daily use, such inflation means may be connected to the therapeutic compression apparatus by, for example, a hose or a tube, which provides inflation to the bladder within the therapeutic compression apparatus and/or wrap. In another embodiment of the present invention, the wound dressing compression system includes an inflation means which can switch between constant static pressure levels (different pressure levels such as 20 mm-Hg, 30 mm-Hg, 40 mm-Hg, up to 200 mm-Hg, etc.) and intermittent varying pressure levels, whereby the pressure is applied to the therapeutic compression apparatus via a hose or tube. The inflation means may be capable of sequentially inflating each bladder separately, and then deflating, or repeating the inflation and deflation as required.

In other embodiments the inflation means is connected to two or more tubes or connectors and thereby connected to two or more bladders, whether multiple bladders within one therapeutic compression apparatus or a single bladder within multiple therapeutic compression apparatuses, or a combination thereof. In another embodiment the inflation means includes one pump with a manifold having multiple chambers each corresponding to a bladder within the apparatus or multiple apparatus, such that the inflation means connected to the manifold is connected to multiple hoses which then connected to each separate bladder, for sequential inflation and deflation of each separate bladder. In another embodiment the system includes one or more sensors to monitor movement of the therapeutic compression apparatus, pressure levels, blood pressure of the patient, tonometry of the target limb, or other sensor data. These and other aspects of the contacts of the subject invention will become more readily apparent from the following description taken in conjunction with the drawings.

### BRIEF DESCRIPTION OF THE DRAWINGS

So that those having ordinary skill in the art to which the subject invention pertains will more readily understand how to make and use the apparatuses of the subject invention, preferred embodiments thereof will be described in detail herein below with reference to the drawings, wherein:

FIG. 1 is an embodiment of the present invention of a lower leg pre-formed absorbent wound dressing over a foot pre-formed absorbent wound dressing on a right foot and leg;

FIG. 2 is an embodiment of the present invention of a lower leg pre-formed absorbent wound dressing over a foot and toe spacer pre-formed absorbent wound dressing on a right foot and leg;

FIG. 3 is a back right side perspective view of the lower leg pre-formed absorbent wound dressing in an opened position;

FIG. 4 is a back left side perspective view of the inventive lower leg pre-formed absorbent wound dressing if FIG. 3 in a closed position;

FIG. 5 is an exploded view of one embodiment of the present invention of a lower leg pre-formed absorbent wound dressing;

FIG. 6 is an exploded view of another larger embodiment of the present invention of a lower leg pre-formed absorbent wound dressing;

FIG. 7 is a front perspective view of another embodiment of the present invention of a lower leg pre-formed absorbent wound dressing in an open position;

FIG. 8 is a front perspective view of another embodiment of the present invention of a lower leg pre-formed absorbent wound dressing in an open position

FIG. 9 is a front right side perspective view of another embodiment of the present invention of a lower leg pre-formed absorbent wound dressing in an open position

FIG. 10 is a back right side perspective view of another embodiment of a lower leg pre-formed absorbent wound dressing in an opened position;

FIG. 11 is front left side perspective of a foot pre-formed absorbent wound dressing on a right foot in the closed position;

FIG. 12 is a back right side perspective of the inventive foot pre-formed absorbent wound dressing of FIG. 11;

FIG. 13 is a front right side perspective view of the inventive foot pre-formed absorbent wound dressing of FIG. 11 in a close position not on a foot;

FIG. 14A is an outer view of the inventive foot pre-formed absorbent wound dressing of FIG. 11 during manufacture before it is connected at the portion around the heel of the foot;

FIG. 14B is an inner view of the inventive foot pre-formed absorbent wound dressing of FIG. 13;

FIG. 15 is an exploded view of the inventive foot pre-formed absorbent wound dressing of FIG. 13;

FIG. 16 is a top view of the inventive foot pre-formed absorbent wound dressing of FIG. 11 in an open position not on a foot;

FIG. 17A is an outer view of an embodiment of the present inventive of foot and toe spacer pre-formed absorbent wound dressing for use on a right foot, with the left foot being a mirror image;

FIG. 17B is an inner view of an embodiment of the present inventive of foot and toe spacer pre-formed absorbent wound dressing for use on a left foot, with the right foot being a mirror image;

FIG. 21 is a perspective front view of the present invention of a foot and toe spacer wound dressing of FIG. 17A in use on a right foot in a closed position with the larger portion on the arch top of the foot, with the left foot being a mirror image

FIG. 22 is a bottom view of the inventive foot and toe spacer pre-formed absorbent wound dressing of FIG. 21;

FIG. 23 is another embodiment of the present invention of a foot and toe spacer pre-formed absorbent wound dressing for use on a left foot, with FIG. 23A is a bottom view and FIG. 23B is a top inner view, with the right foot being a mirror image;

FIG. 24 is an exploded view of the inventive foot and toe spacer pre-formed absorbent wound dressing of FIG. 23A;

FIG. 25 is an embodiment of the present invention of a head pre-formed absorbent wound dressing in use on a head in the closed position;

FIG. 26 is another embodiment of the present invention of a head pre-formed absorbent wound dressing in use on a head in the closed position;

FIG. 27A is an inner view of the inventive foot pre-formed absorbent wound dressing of FIG. 25 during manufacture before it is connected at the portion around the back of the head and FIG. 27B is an outer view of the inventive foot pre-formed absorbent wound dressing of FIG. 27A;

FIG. 28 is exploded view of the inventive head pre-formed absorbent wound dressing of FIG. 25;

FIG. 29 is an embodiment of the inventive lower arm pre-formed absorbent wound dressing and an upper arm pre-formed absorbent wound dressing including an elbow support for use on a left arm, with the right arm being a mirror image; and

FIG. 30 is front left side perspective of the inventive compression system including an elevation member, a zinc oxide gauze dressing, the inventive lower leg pre-formed absorbent wound dressing, a therapeutic compression apparatus, for use on a right leg.

### DETAILED DESCRIPTION OF THE PREFERRED EMBODIMENTS

Preferred embodiments of the subject invention are described below with reference to the accompanying drawings, in which like reference numerals represent the same or similar elements. One of ordinary skill in the art would appreciate that while the apparatuses discussed herein relate to compression therapy of the leg and foot, the scope of the invention is not limited to those exemplary applications and may be sized and shaped for the anatomical portion for which compression therapy is needed.

The subject invention provides a wound dressing used alone or with compression to a patient's limbs, including the extremities, including for example, the foot, ankle and/or leg of a user, in a manner that is simpler, less bulky, more practical, more mobile, and more convenient than current systems. Any limb or body part may be subject to the wound dressing alone or by the instant system including a therapeutic compression apparatus such as for instance a foot, calf, thigh, knee, leg, hip, buttocks, waist, torso, ribs, shoulder, arm, band, fingers, neck, head or the like.

The subject invention provides system for providing a pre-formed absorbent wound dressing alone or with compression to control bleedings, remove and manage exudate from the wound site, and if employing the compression apparatus to also prevent swelling of a limb such as for instance the foot, ankle, lower leg, calf, knee, and/or thigh using a pre-formed wound dressing including an inner hydrophilic wound site contact layer, a middle absorbent layer, and an outer non-absorbent waterproof hydrophobic moisture vapor transfer layer with a support or shaping means to provide the pre-formed shape of the target limb or extremity. In addition, a non-elastic wrap and bladder can be used for compression, which apparatus has at least one and preferably more than one separate bladders within the apparatus, and is connected to an inflation means including a dual means having both constant static pressure levels and varying intermittent pressure levels and the bladders are inflated sequentially. The system is provided in in a manner that allows for consistent measuring of the pressure supplied, as well as safe, comfortable, more practical, more mobile, convenient, effective, and self-application by the patient of the wound dressing.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either both of those included limits are also included in the invention. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications mentioned herein are incorporated herein by reference to disclose and describe the methods and/or materials in connection with which the publications are cited.

It must be noted that as used herein and in the appended claims, the singular forms “a”, “an,” and “the” include plural referents unless the context clearly dictates otherwise. Thus, for example, reference to “a stimulus” would include a plurality of such stimuli and reference to “the signal” would include reference to one or more signals and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may differ from the actual publication dates, which may need to be independently confirmed.

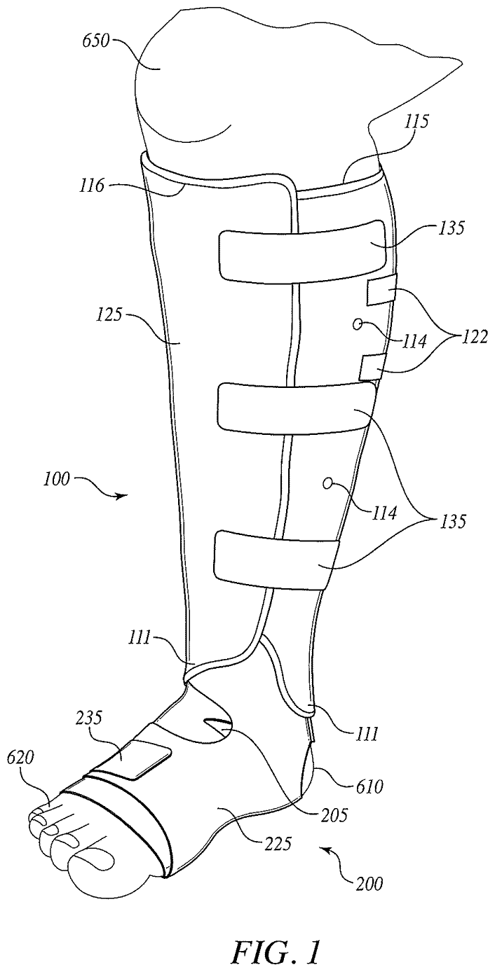

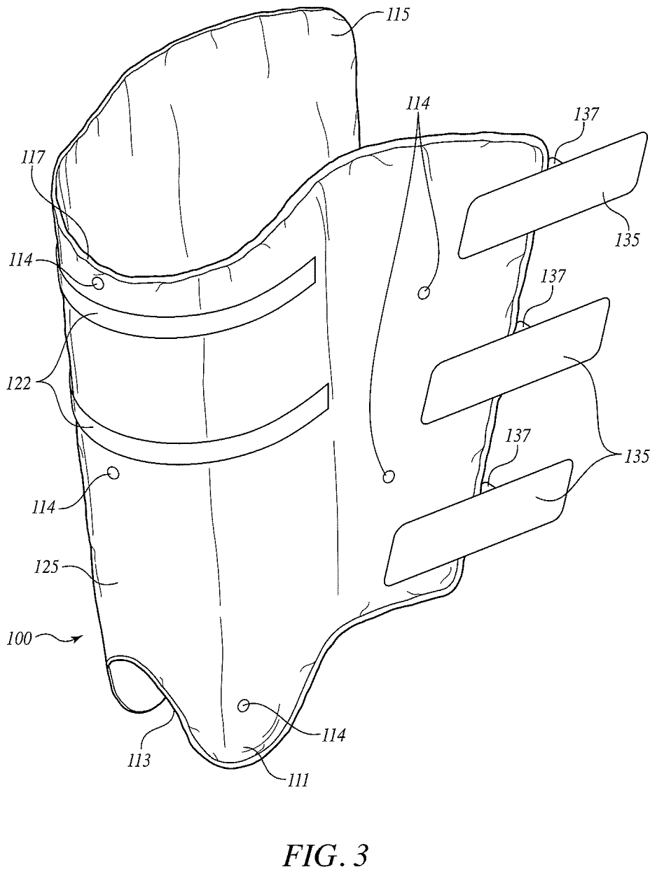

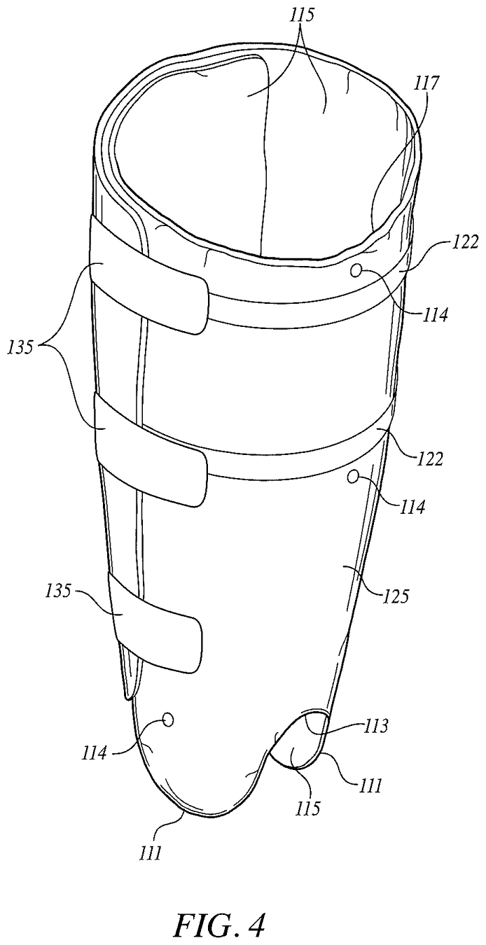

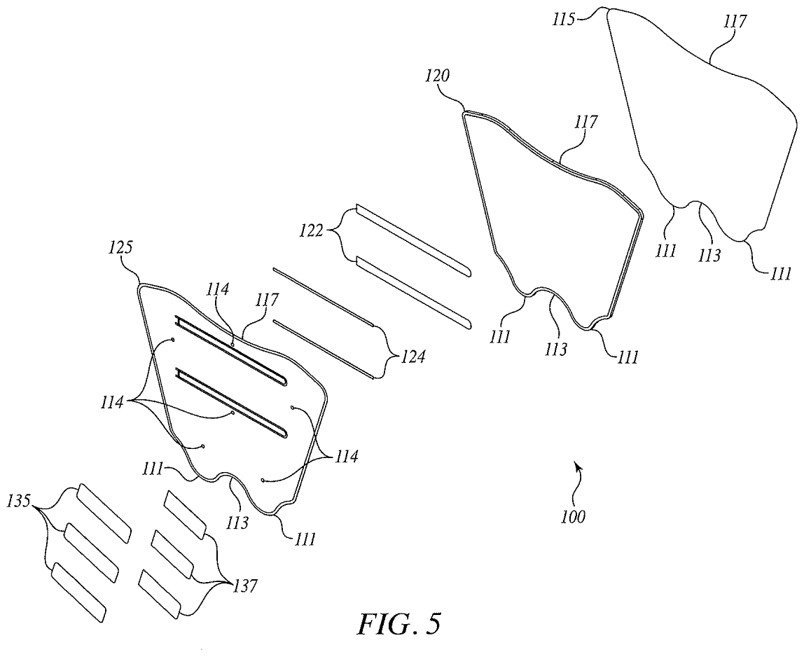

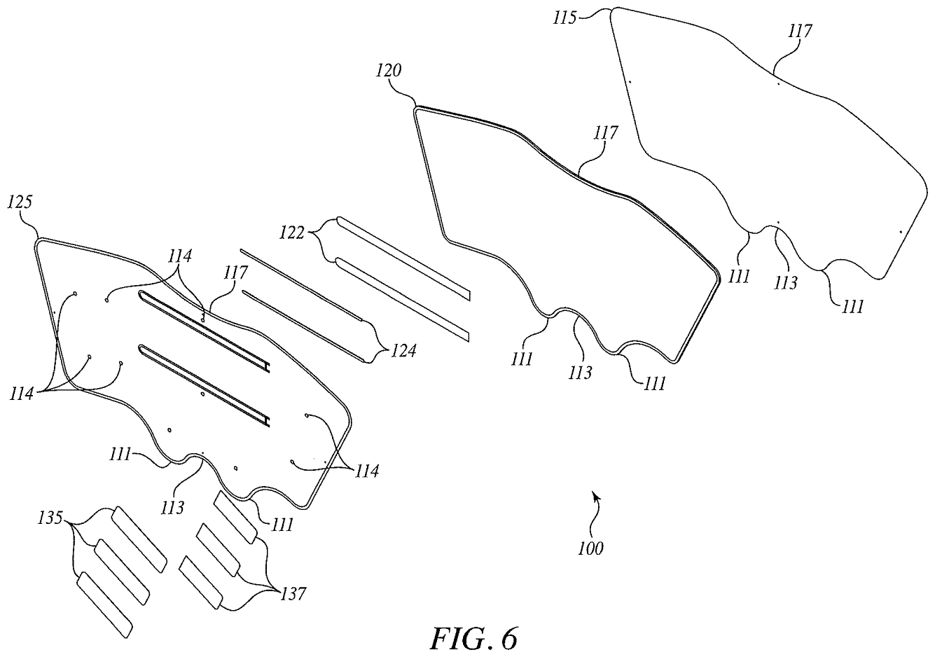

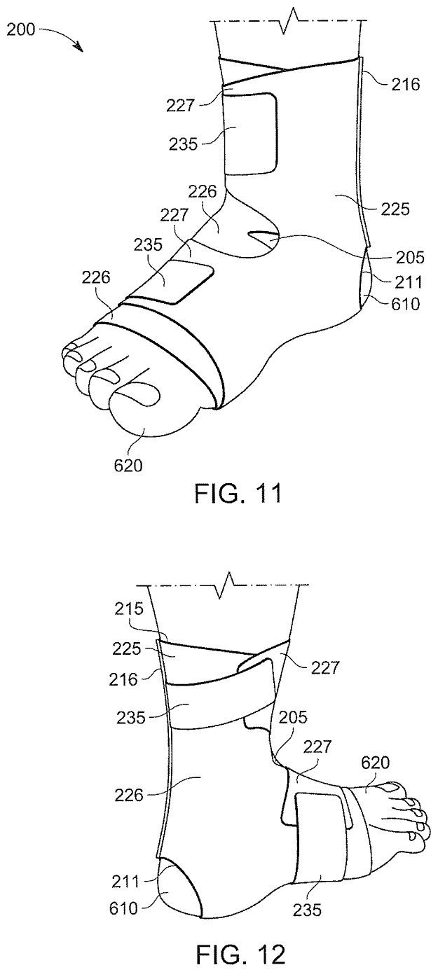

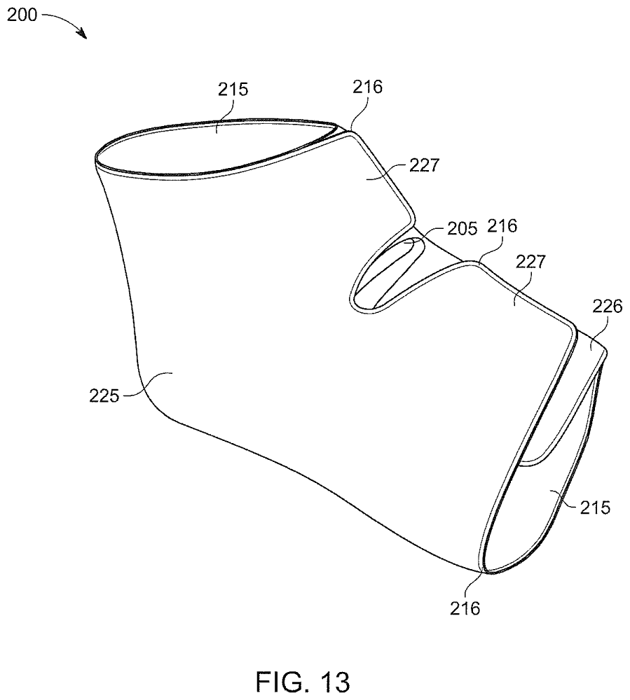

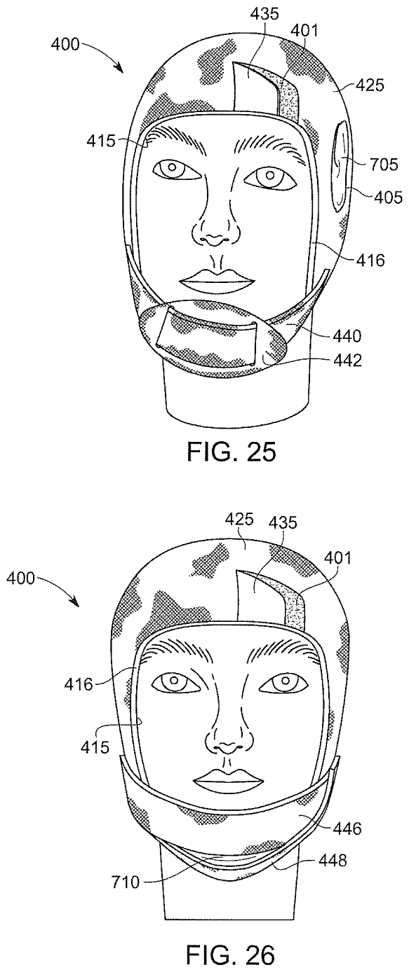

Referring now to FIGS. 1-10, there is illustrated an exemplary embodiment of the inventive pre-formed wound dressing 100 for a lower leg including an inner layer 115 which has contact with the wound site and skin of the patient or user. Other embodiments of the inventive pre-formed absorbent wound dressing include two different foot embodiments 200, 300 as shown in FIGS. 11-21, a head embodiment 400 as shown in FIGS. 25-28, and two different arm embodiments 500, 600. The inner absorbent layer 115, 215, 315, 415, 515, 615 may be comprised of a polymer, cotton or any material compatible with human skin and wounds. The inner wound contact layer 115, 215, 315, 415, 515, 615 may be a hydrophilic material such as cotton, linen, foam, sponge, fibers, and other suitable materials that can absorb exudates and other moisture from the wound as well as hydroconductive materials. The inventive pre-formed wound dressing 100, 200, 300, 400, 500, 600 includes an outer layer 125, 225, 325, 425, 525, 625 which may be in contact with the clothes of the patient or user or a couch, chair or bed linens if the patient or user is non-ambulatory. The outer layer 125, 225, 325, 425, 525, 625 is preferably non-absorbent or less-absorbent. The outer layer 125, 225, 325, 425, 525, 625 may be a hydrophobic material or even waterproof, but should also be able to transfer moisture vapor. The outer layer 125, 225, 325, 425, 525, 625 may be comprised of materials such as paper, film, polymers, rubber, metal and the like. The inventive pre-formed wound dressing 100, 200, 300, 400, 500, 600 includes a middle or central absorbent layer 120, 220, 320, 420, 520, 620 which can absorb exudates and other moisture from the wound and traps such exudate in the middle layer.

The inner porous layer 115, 215, 315, 415, 515, 615 may be comprised of any material compatible with human skin and wounds such as for instance a polymer, a nonwoven polyester, fibers, and the like. The inner layer 115, 215, 315, 415, 515, 615 may be a hydrophilic layer or a hydroconductive layer. The inner porous layer 115, 215, 315, 415, 515, 615 may be flexible, porous, and non-toxic. As used herein, the term “flexible” is meant to cover a range of materials, which exhibit one or more properties such as being flexible, compliant, elastic, or memory retentive. For example, “flexible” is also meant to refer to materials that exhibit elastic or memory properties, i.e., the ability for the material to return to its original shape when stresses applied thereto are reduced or eliminated. The flexible material is preferably flexible or compliant, to allow the flexible substrate to be placed on the desired surface (such as skin, organ, tissue, or the like) in a manner that allows the flexible substrate to conform to the topology of the desired surface. Likewise, the flexible material is preferably porous, to allow the exudate from the wound site, cut, gash, burn, ulcer and the like to pass through or permeate through the flexible material and be trapped in the central absorbent layer 120, 220, 320, 420, 520, 620. By “porous” is meant herein either that the bulk of the inner porous layer 115, 215, 315, 415, 515, 615 has pores, such that the exudate from the ulcer or wound site is soaked up or passed through holes or voids in this inner porous layer 115, 215, 315, 415, 515, 615. For example, the inner layer 115, 215, 315, 415, 515, 615 may also be a wicking layer would be in contact with the wound and skin and may be comprised of a porous material for transfer of the exudates and moisture from the wound to the sandwiched absorbent layer 120, 220, 320, 420, 520, 620. The inner wicking or porous layer 115, 215, 315, 415, 515, 615 permits the transmission of wound fluids and moisture vapor from the wound and skin down into the middle absorbent layer 120, 220, 320, 420, 520, 620. The inner porous layer 115, 215, 315, 415, 515, 6155 may have perforations to assist in the transfer of the exudates and moisture into the inner and then into the middle absorbent layer 120, 220, 320, 420, 520, 620. The materials of the porous layer 115, 215, 315, 415, 515, 615 may be woven or non-woven fabrics, polymers, films and the like. The inner hydrophilic layer 115, 215, 315, 415, 515, 615 may be comprised of any material that transfers moisture such as woven and nonwoven materials (e.g. a nonwoven web of fibers), polymeric materials such as apertured plastic films, e.g. apertured formed thermoplastic films and hydroformed thermoplastic films; porous foams; reticulated foams; reticulated thermoplastic films; and thermoplastic scrims. Suitable woven and nonwoven materials can be comprised of natural fibers or from a combination of natural and synthetic fibers. Examples of suitable synthetic fibers which may comprise all or part of the inner layer 115, 215, 315, 415, 515, 615 include but are not limited to polyamide (e.g. nylon), acrylic (e.g. polyacrylonitrile), aromatic polyamide (e.g. aramide), polyolefin (e.g. polyethylene and polypropylene), polyester, butadiene-styrene block copolymers, natural rubber, latex, spandex (polyurethane) and combinations thereof.

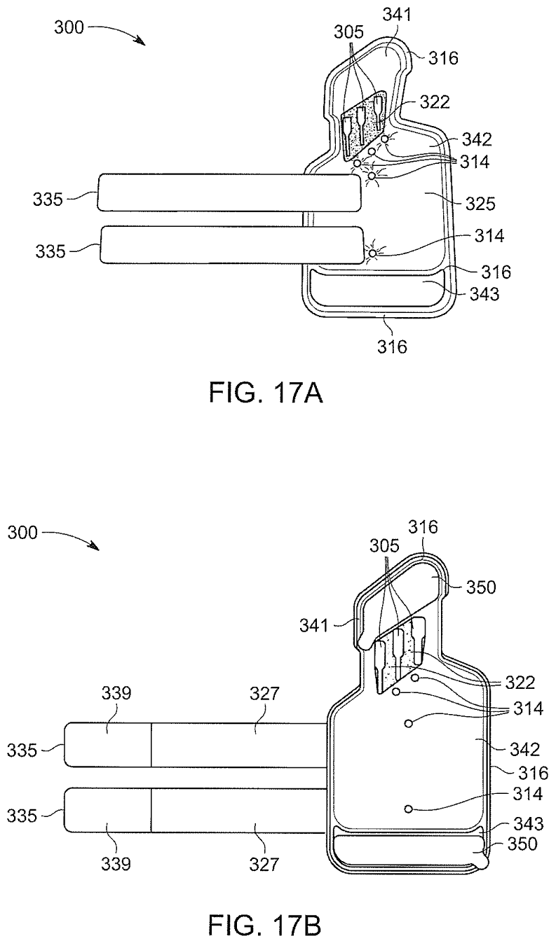

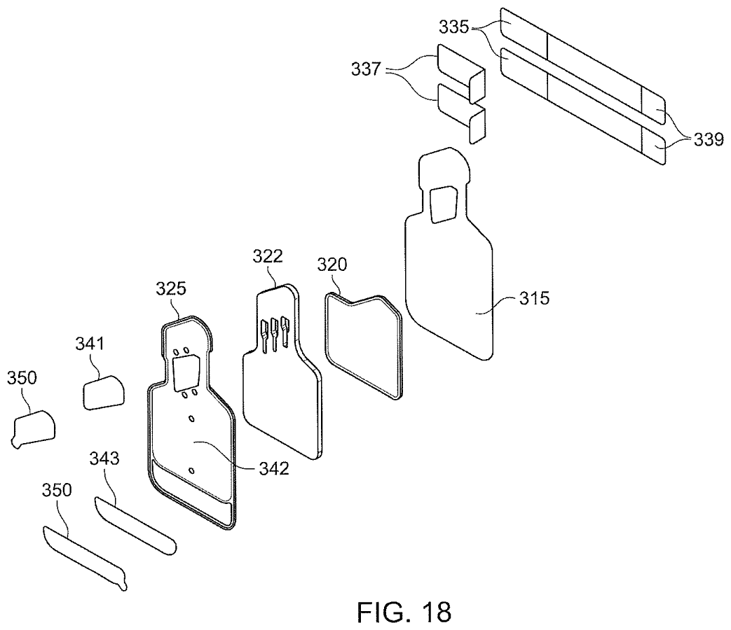

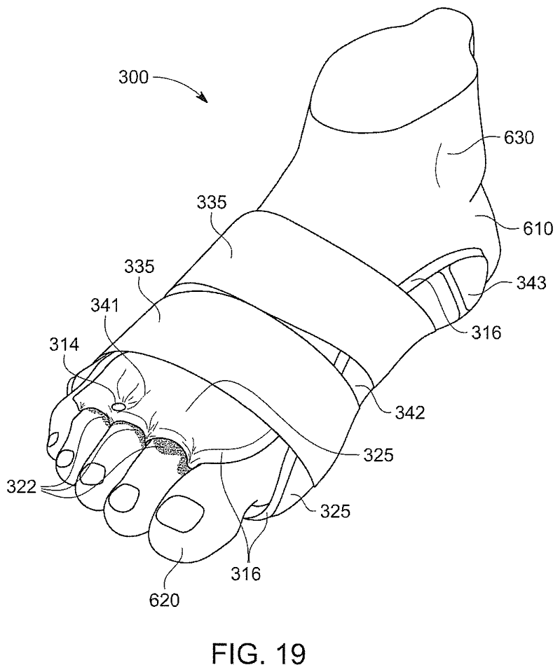

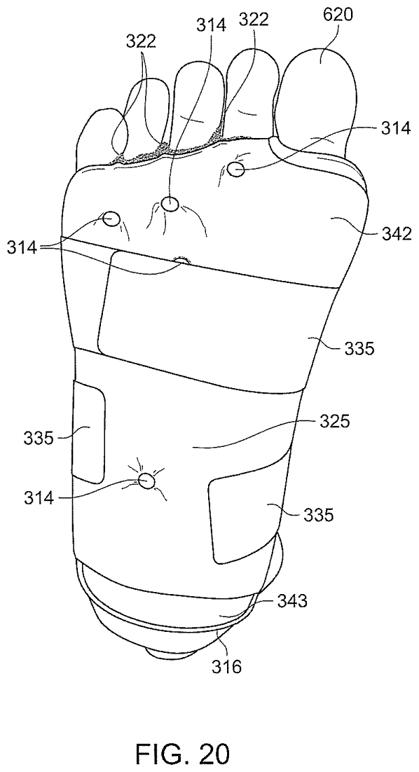

The middle absorbent layer 120, 220, 320, 420, 520, 620 may be a cotton, polymer, and the like having absorption properties and functionality. The inner layer 115 and/or the middle absorbent layer 120 may itself be comprised of multiple layers such as wicking layer, absorbent layer, gel layer, and any combination of layers than can absorb and trap exudate from a wound. The middle absorbent layer 120, 220, 320, 420, 520, 620 may be a hydrophilic material such as cotton, linen, foam, sponge, fibers, and other suitable materials that can absorb and trap exudates and other moisture from the ulcer or wound site. The middle absorbent layer 120, 220, 320, 420, 520, 620 may be cotton, pulp, cellulose, cellulose derivatives, polymers, high molecular water absorbent polymers, thermoplastic resin, cellulosic fiber, acrylic (salt) polymer, foam, woven or nonwoven material selected from the group consisting of rayon, polyester, polyurethane, polyolefin, orlon, hydrogel polymeric material, and combinations thereof. A second absorbent layer 322 as shown in FIG. 17 for the foot embodiment including toe spacers which adds absorption levels and also cushioning to the ball of the foot when worn mostly under the foot such as on the sole of the foot, or cushioning on the arch of the foot when worn mostly on the top of the foot. The central absorbent layer 120, 220, 320, 420, 520, 620 may be comprised of cotton, linen, foam, any material with plurality of open cells and may include at least one of polyurethane, silicone, polyethylene, and gauze. Further, the absorbent layer may be comprised of a comprise a wide variety of liquid-absorbent materials commonly used in disposable diapers, incontinence products and other absorbent articles such as comminuted wood pulp, which is generally referred to as air felt or fluff. Examples of other suitable absorbent materials include creped cellulose wadding; melt blown polymers, including co-form; chemically stiffened, modified or cross-linked cellulosic fibers; tissue, including tissue wraps and tissue laminates, absorbent foams, absorbent sponges, superabsorbent polymers (such as superabsorbent fibers), absorbent gelling materials, or any other known absorbent materials or combinations of materials. Examples of some combinations of suitable absorbent materials are fluff with absorbent gelling materials and/or superabsorbent polymers, and absorbent gelling materials and superabsorbent fibers etc.

The inventive pre-formed absorbent wound dressing 100, 200, 300, 400, 500, 600 includes an outer layer 125, 225, 325 425, 525, 625 which may be in contact with the sock or clothes of the patient or user or a couch, chair or bed linens if the patient or user is non-ambulatory. The outer layer 125, 225, 325 425, 525, 625 is preferably non-absorbent or less-absorbent. The outer layer 125, 225, 325 425, 525, 625 may be a hydrophobic material. The outer layer 125, 225, 325 425, 525, 625 may be comprised of materials such as paper, film, polymers, laminate, rubber, metal and the like. Not shown but envisioned is a layer on the outer materials which indicates when the inner porous layer 115, 215, 315, 415, 515, 615 or central absorbent layer 120, 220, 320, 420, 520, 620 has reached a moisture level that the entire absorbent foot wound dressing 100 should be replaced such as for example a hydrochromic ink or other indicator. The outer layer 125, 225, 325, 425, 525, 625 may also be an outer waterproof vapor transfer layer that is moisture permeable and liquid impermeable. Suitable materials include hydrophobic materials such as by way of example polymers, woven and non-woven materials, films, laminates and combinations thereof. Further, the outer layer 125, 225, 325 425, 525, 625 prevents the exudates absorbed by the inner and/or absorbent layers 115, 120 and prevent contact of the exudate with clothing, linens, bed sheets, furniture and the like. The outer layer 125, 225, 325 425, 525, 625 may be substantially impervious to liquids (e.g., blood, pus, etc.) and comprises a laminate of a nonwoven and a thin plastic film such as a thermoplastic film. Other suitable outer layer 125, 225, 325 425, 525, 625 materials may include breathable materials that permit vapors to escape from the inventive pre-formed wound dressing 100, 200, 300, 400, 500 while still preventing exudates from passing through the outer layer 125, 225, 325 425, 525, 625. Exemplary breathable materials may include materials such as woven webs, nonwoven webs, composite materials such as film-coated nonwoven webs, and microporous films. Not shown but envisioned is a layer on the outer materials which indicates when the inner layer or middle layer has reached a moisture level that the entire pre-formed absorbent wound dressing should be replaced. Other embodiments are envisioned that allow the user to know when to replace parts of the system.

The inventive pre-formed absorbent wound dressing 100, 200, 300, 400, 500, 600 include a securing means 130, 230, 330, 430, 530, 630 for placing and securing the pre-formed absorbent wound dressing to the selected or target body part. These embodiments also include a securing means 130 by way of non-limiting example only as fasteners or tabs 135, 235, 335, 435, 440, 446, 448, 535, 635. The fasteners or tabs 135, 235, 335, 435, 440, 446, 448, 535, 635 have in one embodiment an adhesive (not shown) and a portion 137, 237, 337, 437, 537, 637 which can be peeled or removed from the inner surface of the fasteners or tabs 135, 235, 335, 435, 535, 635 to expose or release the adhesive so that the fasteners or tabs 135, 235, 335, 435, 535, 635 can secure one side of the outer layer 125, 225, 325 425, 525, 625 of the inventive pre-formed absorbent wound dressing 100, 200, 300, 400, 500, 600 when closed on the user's selected body part or in another embodiment have a loop and hook connection such as 401, 411, 431, 440, 446, 448. Other securing means 130 may be employed such as hook and loop, buttons, zippers, fasteners and other means of securing the inventive pre-formed absorbent wound dressing 100, 200, 300, 400, 500, 600 onto the user's selected or target body part, limb or extremity. In certain embodiments the distal end of the fasteners or tabs 135, 235, 335, 435, 535, 635 include a portion without any adhesive 139, 239, 339, 439, 539, 639 (not all shown) so that the user can pull off the fastener or tab 135, 235, 335, 435, 535, 635 to open the pre-formed absorbent wound dressing 100, 200, 300, 400, 500, 600 to check the wound site. Thus the user only has to have one hand to grip and pull off the fastener or tab 135, 235, 335, 435, 535, 635 making it easier to view and check the wound site for healing or complications. Further in some embodiments of the inventive pre-formed absorbent wound dressing there is one fastener or tab, or two fastener or tabs, or three or more fasteners or tabs, depending on the body anatomy of the selected or target body part. The adhesive for the fastener or tabs 135 may be comprised of a material that maintains it adhering properties even when opened and closed repeatedly, so that the user can remove the pre-formed wound dressing 100, 200, 300, 400, 500 to engage in a activities such as bathing and then replacing the pre-formed wound dressing back onto the wound site after completing a bath or shower. This importantly allows the patient or user to have a lifestyle not affected by their wound dressing, compared to common conventional compression bandages and plasters, etc.

Turning to the lower leg embodiment 100 of the pre-formed absorbent wound dressing as shown in FIGS. 1-10, there is a distal portion 111 configured for the user's ankle 630 on the foot as well as an opening aperture or curve 113 for the bony back section of the ankle as well as a proximal portion 117 configured for better wear at the user's calf nearer the knee 650. Thus the lower leg embodiment 100 of the pre-formed absorbent wound dressing is configured to the shape of the lower leg as the selected or target body part. Based on this shape configuration, the inventive lower leg embodiment 100 of the pre-formed absorbent wound dressing fits better onto the lower leg based on the lower leg anatomy, this assisting in healing of the wound. The lower leg embodiment 100 of the pre-formed absorbent wound dressing also includes certain weld spots 114 and weld lines 116 to better shape the inventive lower leg embodiment 100, though other non-welded sections are contemplated for shape and fit.

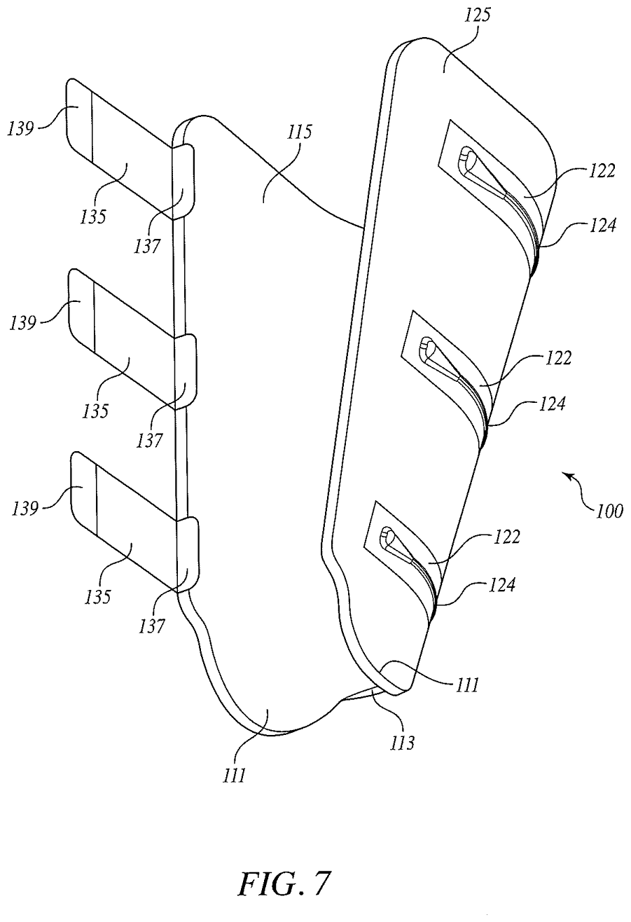

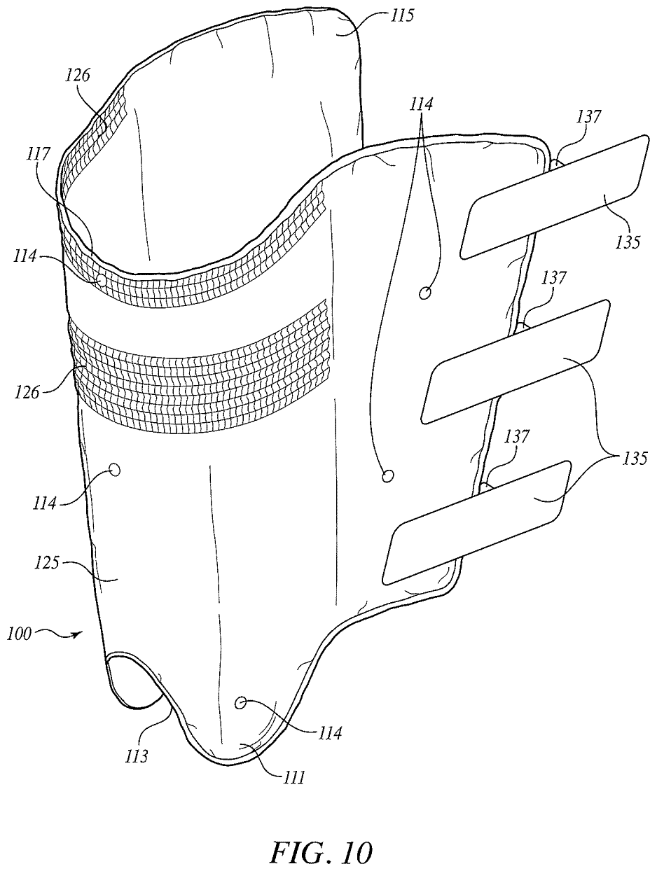

In these embodiments of the lower leg 100 pre-formed absorbent wound dressing there are also a shaping or support means 124 such as plastic, wires, elastic or any other material capable of shaping and/or supporting the inventive pre-formed absorbent wound dressing 100 into a cone shape for the lower leg. Three non-limiting shaping or supporting means 124 are shown but other may be employed, such as plastic in FIGS. 1-6, wires in FIG. 7 and elastic in FIG. 10. The shaping means such as the wires 124 are bent into the required shape, in this example a cone. The shaping means such as wires 124 are secured to the outer layer 125, 225, 325 425, 525, 625 via a means such as a strip 122 including adhesive during the manufacturing process so that the strip 122 is welded on three sides and then the wire 124 placed within the created channel and then welded on the fourth side and in this embodiment is within the inventive lower leg pre-formed absorbent wound dressing 100 in between the middle absorption layer 120 and the outer hydrophobic moisture vapor transfer layer, though another embodiment is shown in FIGS. 1-2 and 7. In this embodiment and by way of non-limiting example, the inventive lower leg pre-formed absorbent wound dressing 100 may be manufactured or created by die-cutting the outer layer 125, middle absorbent layer 120 and inner layer 115 as separate operations. Then all layers are aligned on pins and RF welded together via three channels and the outer layer 125 to the shaping cover 122. Further the layers are aligned and then RF welding it to the perimeter, outer layer 125 to inner layer 115 with the middle absorbent layer 120 in between. In this embodiment then the three curved wires or bands 124 are inserted into the three channels followed by ultrasonic welding to the three channel ends to close the channels and constrain the wires 124 or bands. Finally, in this embodiment the three sets of fasteners or tabs 135, 135, 135 having adhesive on the underside and release liners 137, 137, 137 are added, notably the adhesive is capable of maintaining a level of adhesion to be opened and closed repeatedly.

Other shaping means may be employed such as plastics, wood, metals, polymers, films, alloys that have a shape memory such as nitinol, and the like. In other embodiments as shown in FIG. 1-4 the shaping or support means are plastic that are placed in the layers flat and then rolled up in packing and transport so that the plastic 124 takes on a curved shape, sometimes referred to as plastic creeping wherein the plastic deforms permanently under the influence of constant stress: tensile, compressive, shear, or flexural. In the other embodiment shown in FIG. 10, the shaping or support means is two elastic bands 126 formed at the top of the lower leg pre-formed absorbent wound dressing and further down on the calf portion, though it is contemplated that there could only be one elastic portion 126, or three or more elastic portions as needed for the selected or target body part if not a lower leg, such as a thigh, torso, hip, chest, shoulder, arm, elbow, wrist, hand, neck or head. In the embodiment shown in FIG. 7, the shaping means of the wires 124 are secured to the outer layer 125 via a paper strip 122 including an adhesive layer (not shown). The securing means 130 is a series of three fasteners 135 having an adhesive strip beneath the releasing strip 137.

On the inventive lower leg pre-formed absorbent wound dressing 100, the proximal portion 116 of the inner layer 115 and matching outer layer 125 is configured for better placement along the user's calf or knee 650 on the leg and on the distal end has a distal portion 111, 113 configured for better placement along the user's ankle 630 on the foot. These portions are shaped so that when the inventive lower leg pre-formed absorbent wound dressing 100 is secured on the lower leg there is no gap between the calf or knee 650 or along the ankle 630 above the foot. Other configurations and shapes may be employed depending on the limb where the inventive pre-formed absorbent wound dressing 100 is being placed, such as the thigh, knee, foot, arch, torso, arm, hand, elbow, shoulder, chest, neck and other body parts. The configurations shown are non-limiting examples of the lower leg limb in these embodiments.

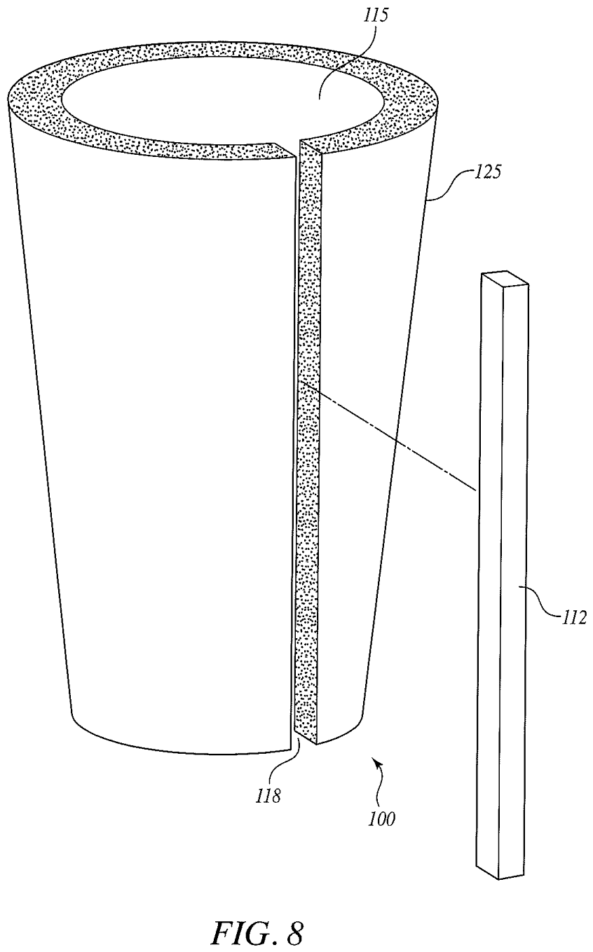

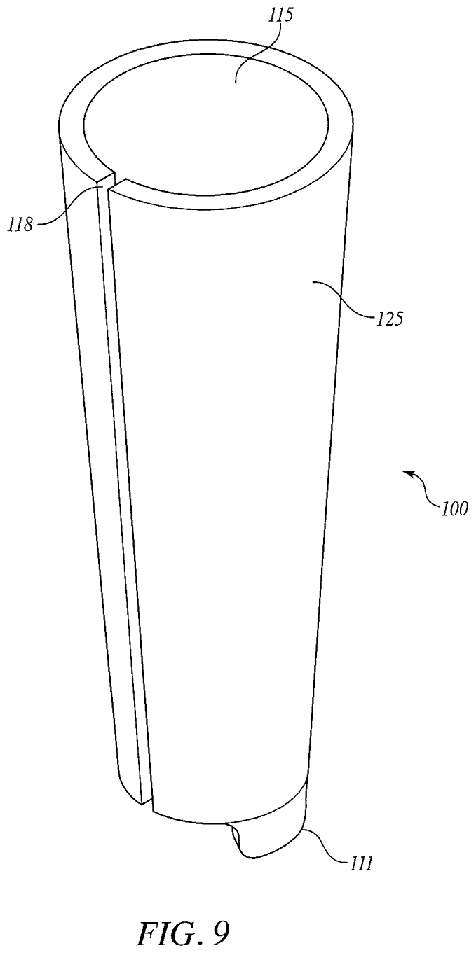

The embodiment shown in FIGS. 8 and 9 is a pre-formed absorbent wound dressing 100 in the shape of a cone including a slit or opening 118 which is somewhat flexible so as to increase the opening 118 in order to place the user's lower leg within the inventive pre-formed absorbent wound dressing 100. There is provided a securing means 130, which in this embodiment is a wedge-shaped portion 112 to be placed within the opening slit 118 when the inventive pre-formed absorbent wound dressing 100 is closed onto the user's leg. Other securing means can be used as well including tape, wraps and the like to keep the wedge shaped portion 112 within the opening slit 118 while the inventive pre-formed absorbent wound dressing 100 is placed on the user's lower leg. In another embodiment as shown in FIG. 9, a distal portion 111 is included configured for the user's ankle 630.

Many of the embodiments of the inventive foot pre-formed absorbent wound dressing 200 configured for application on the foot as shown in FIGS. 1, 11-15 are for the right foot of the individual when in use, but the mirror image of the embodiments may be employed for application on the left foot of the individual user (300) and indeed in the embodiments shown the inventive foot pre-formed absorbent wound dressing 200 is ambidextrous and not right or left specific. The inventive foot pre-formed absorbent wound dressing 200, 300 is sized in a range dependent on the target limb such as the width and length of the inventive foot pre-formed absorbent wound dressing 200, 300 may be sized according to known shoe sizes for youth, men, women, etc.

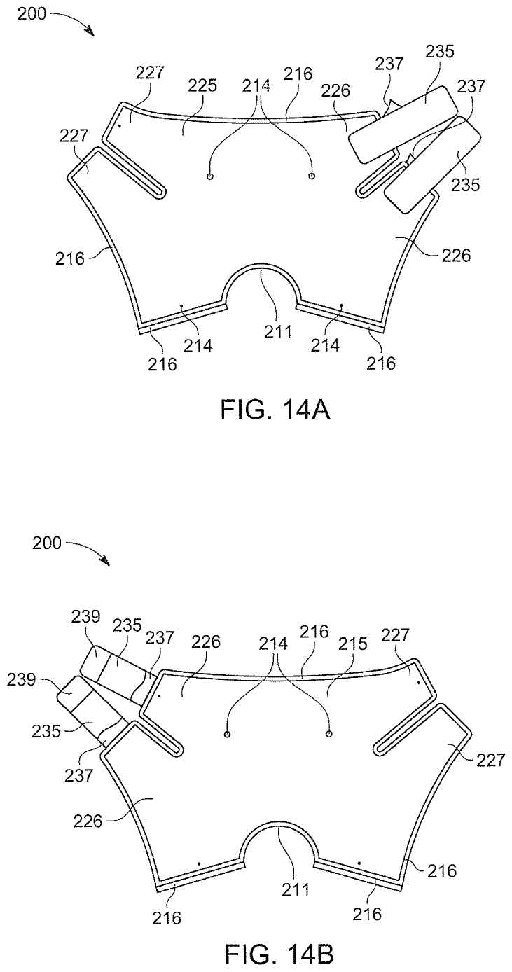

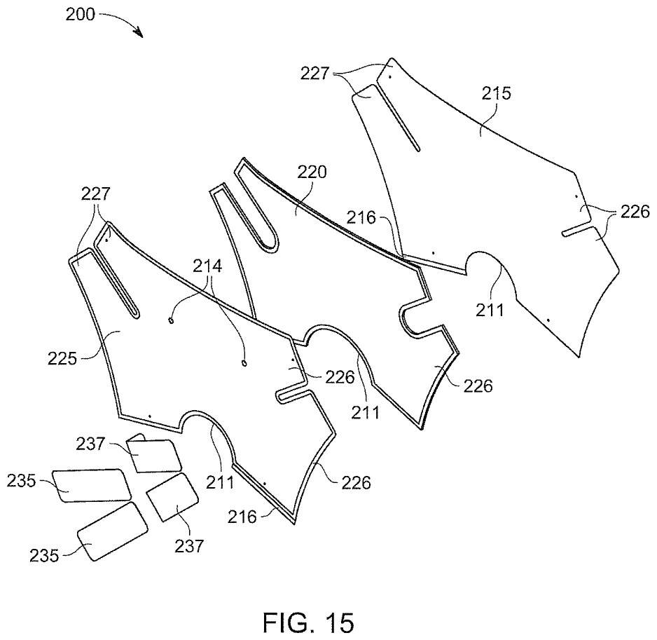

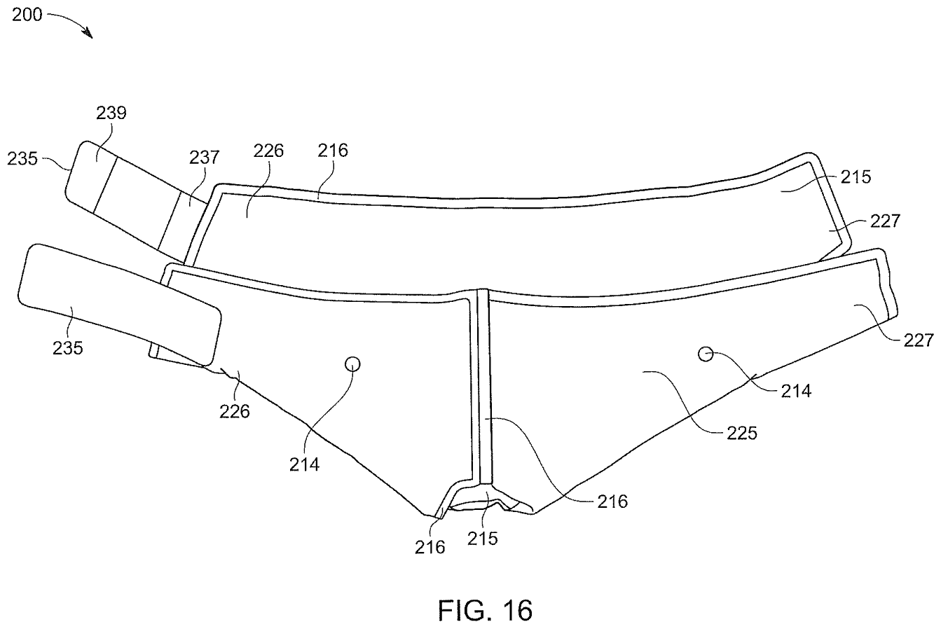

The inventive foot pre-formed absorbent wound dressing 200 may be created or manufactured as shown in the exploded view of FIG. 15 and partially constructed in FIGS. 14A and 14B. By way of non-limiting example of manufacture, the outer layer 225, middle layer 220, and inner layer 215 are die-cut as separate operations. Then all layers are aligned on pins and RF welded together via the outer edges 216 with a multiple of dot welds 214 located through all layers but seen in these Figures on the outer layer 225 configured to create a pre-formed shape of the selected body part of the foot when in use, and including below the opening or apertures 211 for the heel. Additionally, the securing means such as two tabs 235 in this example are added with an adhesive (not shown) and plastic release tabs 237, notably the adhesive or tape is capable of maintain is adhesion to be opened and closed repeatedly. Finaly the two straight parts along the curved section 211 for the heel are welded or joined forming the aperture 211 for insertion of the heel 610 when in use, as shown in FIG. 16 where the manufactured foot pre-formed absorbent wound dressing 200 is folded and ready for use. The shape of each side portion of the inventive foot pre-formed absorbent wound dressing 200 includes one side which has two wide portions 227, 227 including a longer slit, these two positions 227, 227 are placed on the arch of the foot first when in use and then the opposite side has two thinner portions 226, 226 with a shorter slit and each portion 226, 226 has a fastener or tab 235, 235 connected so that the two thinner portions 226, 226 are placed over the two thicker or wider portions 227, 227 and the tab releases 237, 237 are removed activating the adhesive (not shown) under the fastener or tabs 235, 235 and they are adhered to the top of the thinner portion 227, 227 securing the inventive foot pre-formed absorbent wound dressing 200 on the foot. In another embodiment the opposite is shown with the longer thinner portions 227, 227 having the fastener or tab 235, 235 connected so that the two thicker portions 226, 226 are placed on the foot first and then the thinner portions 227, 227 are placed over the two thicker or wider portions 226, 226 and the tab releases 237, 237 are removed activating the adhesive (not shown) under the fastener or tabs 235, 235 and they are adhered to the top of the thicker portion 226, 226 securing the inventive foot pre-formed absorbent wound dressing 200 on the foot. FIGS. 11-13 show the inventive foot pre-formed absorbent wound dressing 200 on a foot with the aperture 211 configured on the heel 610 of the foot and where the portions 226, 226, 227, 227 overlap there is a slight aperture 205, though in other embodiments not shown the overlap may be complete so there is no aperture 205 present.

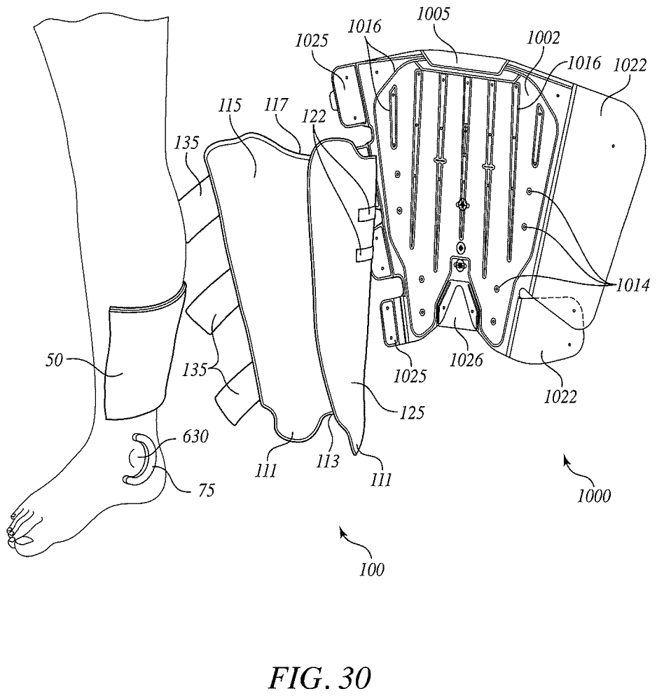

The method of securing the inventive foot pre-formed absorbent wound dressing 200 configured for application to the user's foot includes placing the inventive foot pre-formed absorbent wound dressing 200 on the user's foot with the inner layer 215 against the target ulcer or wound site and the heel 610 of the user placed within the opening or aperture 211 and optionally the inventive pre-formed foot absorbent wound dressing 200 may be placed over an already applied and secured wound dressing 50 and/or elevation apparatus or bolster 75 then the user places the portion without the tabs 235 over the arch of the foot (either 226, 226 or 227, 227) and then places the other side (either 226, 226 or 227, 227) over and activates the adhesive by removing the release tabs 237, 237. In the embodiment shown in FIG. 14B the fastener or tabs 235, 235 including a portion 239, 239 that does not have adhesive so that the user can grip it and pull the fastener or tabs 235, 235 off to open the closed and secured inventive foot pre-formed absorbent wound dressing 200 to check on the wound site and then close it up again. In some embodiments of the method as shown in FIG. 30, an optional gauze wound dressing 50 impregnated with medication, anti-bacterial coating, one or more coatings, silver, or zinc oxide may be placed first on the target ulcer or wound site before the foot pre-formed absorbent wound dressing 200 is placed on the foot. As shown in FIG. 1 a second pre-formed absorbent wound dressing 100 configured for the lower leg is placed on the lower leg below the knee 650 and above the ankle 630 with the inner layer 115 against the skin of the leg and any ulcer or wound site, after the foot pre-formed absorbent wound dressing 200 configured for the foot has been secured on the foot of the user as the lower leg pre-formed absorbent wound dressing 100 overlaps the foot pre-formed absorbent wound dressing 200 around the ankle 630 area and heel area 610. The user places the inner layer 115 against the skin of the leg and any ulcer or wound site (optionally over any wound dressing 50 and/or elevation apparatus or bolster 75 already placed on the skin over an ulcer or wound site) and closes the tabs 135 by activating the adhesive (not shown) by removing the tabs 137. Further, an inventive therapeutic compression apparatus 1000 as shown in FIG. 30 may be placed over all of the inventive pre-formed absorbent wound dressing 100, 200, 300.

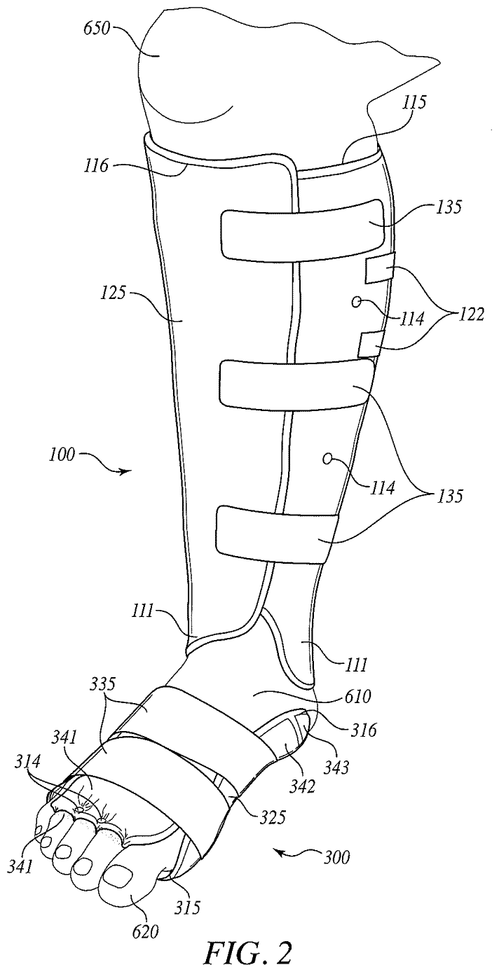

FIGS. 17-21 and FIG. 2 show another embodiment of the present invention of a foot and toe spacer pre-formed absorbent wound dressing 300 which include multiple openings or apertures 305 for placement of the toes of the individual when placed onto the foot and toe spacing 322 which goes between the toes 620 on the foot when in use. This assists in securing the inventive foot and toe spacer pre-formed absorbent wound dressing 300 onto the foot and less movement of the inventive foot and toe spacer pre-formed absorbent wound dressing 300 once placed onto the foot.

The foot and toe spacer pre-formed absorbent wound dressing 300 includes a securing means which as shown includes by way of non-limiting example two fasteners or tabs 335, 335 with such shown as configured to be placed on the ankle 630 or around the foot such as on top of the arch of the foot if the larger portion 342 (also including portion 343) of the foot and toe spacer pre-formed absorbent wound dressing 300 is placed on the bottom of the foot, or the reverse with the second fastener or tab 335 placed on the bottom of the foot if the larger portion 342 is placed on the top of the foot with portion 343 on the front of the ankle 630. Other securing means can be used as well including tape, wraps, buckles, snaps, and the like to keep the foot and toe spacer pre-formed absorbent wound dressing 300 applied on the foot. As shown in FIGS. 17A-17B and 18, the fastener or tabs 335, 335 include an adhesive layer (not shown) for securing the it to the foot of the individual and a portion 350 which can be peeled or removed from the inner surface of the fasteners or tabs 335 to expose or release the adhesive so that the straps 335 can be applied and secured on the foot (top or bottom) or ankle 630 of the foot. Other securing means 330 may be employed such as hook and loop, buttons, zippers, fasteners and other means of securing the foot and toe spacer pre-formed absorbent wound dressing 300 onto the user's foot.

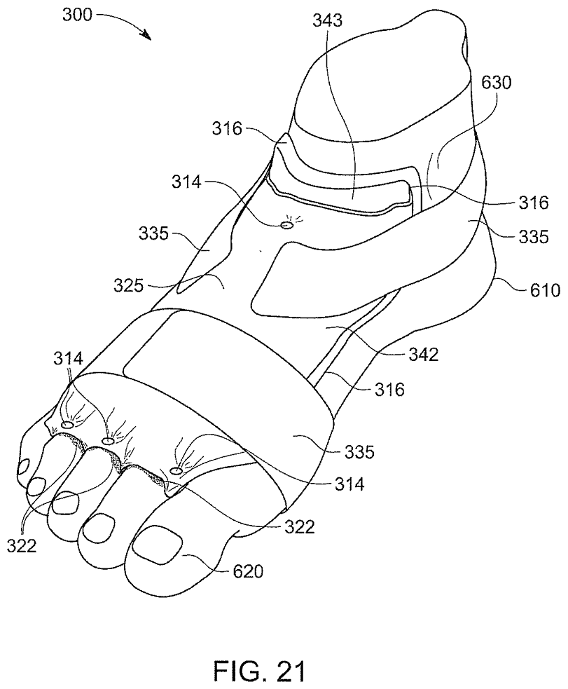

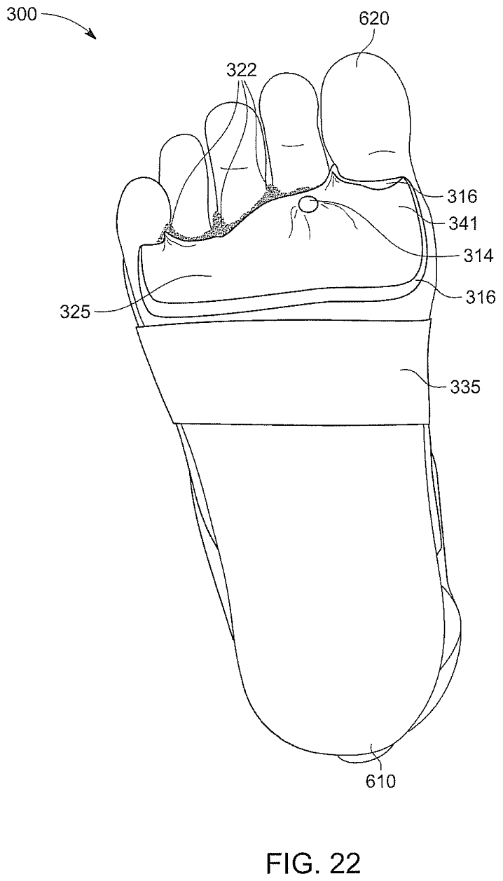

The foot and toe spacer pre-formed absorbent wound dressing 300 is configured in the shape of a foot where in the larger portion 342 can placed under the sole of the foot or on top of the arch of the foot, depending on the location of the target wound site or ulcer, burn, gash, cut or surgical site. The smaller portion 341 is located above the openings 305 when the foot and toe spacer pre-formed absorbent wound dressing 300 is laid flat. The smaller portion 341 will be applied and placed either on top or below the foot depending on the location of the target wound site or ulcer, burn, gash, cut or surgical site. As shown in FIGS. 19-20 the smaller portion 341 will be applied or placed on the top of the foot if the target foot wound site or ulcer, burn, gash, cut or surgical site is located under the foot, and the opposite as shown in FIGS. 21-22 where the smaller portion 341 will be applied on the bottom of the foot (ball of the foot below the toe area) if the target wound site or ulcer, burn, gash, cut or surgical site is located on the top of the foot. The smaller portion 341 includes securing means such as an adhesive layer as shown which is activated or released by removing the paper or film tab 337, 350. The configuration of placement of the fasteners or tabs 335 is shown in different locations of FIGS. 17A-17 and 18 where they are parallel on one side, compared to another embodiment where the location of the fasteners or tabs 335, 335 are perpendicular to each other and one a side and bottom of the foot and toe spacer pre-formed absorbent wound dressing 300. In each embodiment as shown there are release tabs 337, 337, 350 which have to be removed to activated the adhesive layer (not shown) below. Again, other securing means may be employed and if the adhesive layer is employed it typically will not be located so as to adhere to the target foot ulcer or wound site, though in some embodiments not shown that could be employed depending on the adhesive properties or such as a skin substitute or liquid bandage. The adhesive layer is any material compatible with the skin and body of the user. The adhesive layer may be comprised of a material that maintains it adhering properties even when opened and closed repeatedly, so that the user can remove the foot and toe spacer pre-formed absorbent wound dressing 300 to engage in a activities such as bathing and then replacing the absorbent foot wound dressing back onto the foot ulcer or wound site after completing a bath or shower. This importantly allows the patient or user to have a lifestyle not affected by their wound dressing, compared to common conventional compression bandages, casts, and plasters, etc.

Many of the embodiments shown are for the right foot of the individual when in use, but the mirror image of the embodiments may be employed for application on the left foot of the individual user. Given the anatomy of the foot by the toes 620 the current embodiments are specific for a right or left foot, but it is envisioned other embodiments may be ambidextrous and not depending the specific foot chosen. The inventive foot and toe spacer pre-formed absorbent wound dressing 300 is sized in a range so that the larger portion 342 (combined with 343) may be placed either under the foot or on top of the foot. The length of such larger portion 342 (combined with 343) can be as small as just over the ball of the foot, or longer to be placed over the ball through under arch of the foot, or still longer to be placed over the entire underside of the foot, or even longest to be placed on the entire underside of the foot and partially up onto the heel area of the foot. In yet other embodiments the width and length of the larger portion 342 (combined with 343) may be sized according to known shoe sizes.

The inventive foot and toe spacer pre-formed absorbent wound dressing 300 may be created or manufactured by die-cutting the outer layer 335, central layer 320, cushioning and toe spacer layer 322, and inner 315 layer as separate operations. Then all layers are aligned on pins and RF welded together via the outer edges 316 with a multiple of dot welds 314 located on the larger portion 342 including below the opening or apertures 305 for the toes so as to shape and configure the inventive foot and toe spacer pre-formed absorbent wound dressing 300 for the anatomy and shape of the selected and target body part of the toes and foot. Finally, the adhesive layers and fastener or tabs 335, 335 are added, notably the adhesive or tape is capable of maintain is adhesion to be opened and closed repeatedly, as well as the release tabs 337, 337, 350, 350.

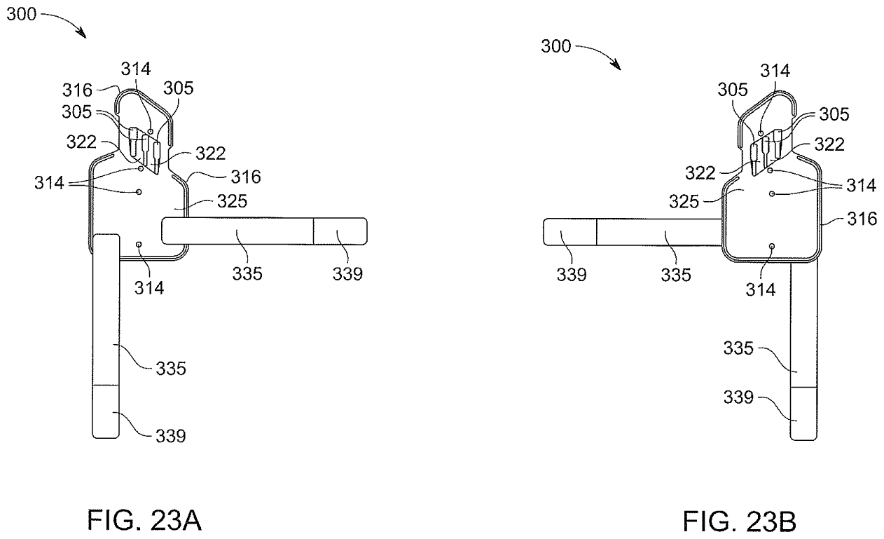

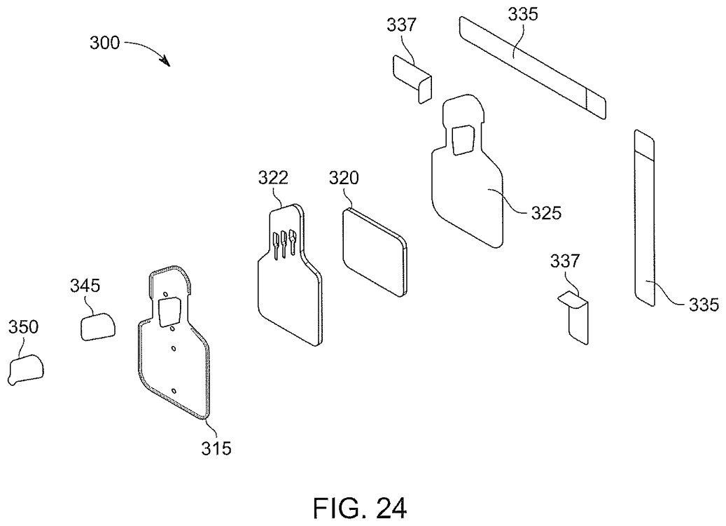

The method of securing the inventive foot and toe spacer pre-formed absorbent wound dressing 300 to the user's foot includes pulling the release tabs 350, 350 to activate the adhesive layer on portions 341, 343 and then placing the larger portion 342 either on the bottom sole or top arch of the foot with the inner porous layer 315 against the target ulcer or wound site, then the user places the toes within the openings 305, finally the adhesive layers 335, 341, 343 are activated by removing the tabs 337, 350. In some embodiments the fasteners and tabs 335, 335 have to be placed either on the back of the heel 610 below or around the ankle 630 of the foot or either above or below the foot being on the opposite side of the foot as the larger portion 342. In some embodiments of the method an optional gauze 50 impregnated with medication, anti-bacterial; coatings, one or more coatings, silver or zinc oxide may be placed first on the target ulcer or wound site before the inventive foot and toe spacer pre-formed absorbent wound dressing 300 is placed on the foot, as well as a metatarsal elevation member or bolster (not shown but disclosed in co-owned application Serial No. PCT/US23/25642 entitled “Elevation Apparatus, System and Methods of Use”, the entirety of within is incorporated herein). The inventive foot and foot/toe spacer pre-formed absorbent wound dressings 200, 300 may be used by emergency personnel on location of a trauma to control bleeding or by clinical and medical professional post-surgery or for treatment and prevention of ulcers and bed sores on the foot, toes, in between the toes, sole, arch, and side of the foot or for post-surgical procedures. The multiple shapes shown in FIGS. 11-24 for configuration of placement on the inventive pre-formed absorbent wound dressing 200, 300 are merely non-limiting examples and other shapes may be employed.

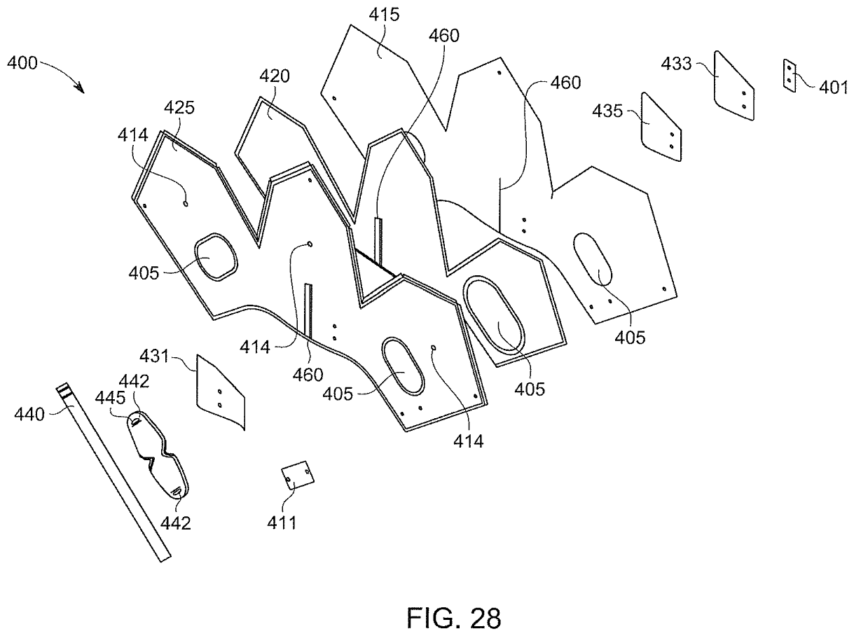

Another embodiment of the present invention is shown in FIGS. 25-28 for use on the head of an individual. The inventive head pre-formed absorbent wound dressing 400 may be used by emergency personnel on location of a trauma to control bleeding or by clinical and medical professional post-surgery or for treatment and prevention of ulcers and bed sores on the back or side off the head or for post-surgical procedures. The inventive head pre-formed absorbent wound dressing 400 is absorbent and includes a retaining or securing means such as a chin strap 435 or adhesive to place the inventive pre-formed wound dressing 400 on the head of the individual for better fit over the wound. The inventive head pre-formed absorbent wound dressing 400 includes two openings or apertures 405, 405 for the ears 705 and optionally additional adjusting means 435. It has three layers as shown in FIG. 28 including the inner porous hydrophilic layer 415, middle absorbent layer 420 and outer hydrophobic vapor transfer layer 425, each having a slit 460 on the top portion through which the adjusting means 431, 435 is connected during manufacture of the inventive head pre-formed absorbent wound dressing 400. The inventive head pre-formed absorbent wound dressing 400 may be created or manufactured by die-cutting the outer layer 425, absorbent layer 420 and inner layer 415, each including the corresponding slit 460, as separate operations. Then by way of non-limiting example only all layers 415, 420, 425 are aligned on pins and RF welded together as well as spot welds 414 strategically chosen based on the configuration of the shape of the selected or target body part, this embodiment being the head. Further the layers are aligned and then RF welded to the perimeter 416 with the outer layer 425 to inner layer 415 having absorbent layer 420 in between. The adjusting mean 435 (including layers 431, 433) which is then partially inserted and in this non-limiting example RF welded into the slit 460 so that a flap is formed that is configured to move and connect to the hook portion 401 with the underside loop portion 431 of the fastener or tab 435. As shown in FIG. 28 the fastener or tab 435 of the adjusting means has multiple layers including a layer 433 comprised of by way of non-limiting example a polymer such as polyethylene net and an outer layer 435 comprised of by way of non-limiting example a polyester or film, and then the fastening or adjusting layer 431 which in this example is a loop material capable of connecting and fastening to the hook layer 401. As shown in FIG. 28 the hook portion 401 is a rectangle compared to the shape as shown in FIGS. 25-26 where is it a trapezoid type of shape, though many other shapes and configurations are possible as long as the fastened means 435 can be moved and connected on the underside loop portion 431 to the hook portion 401. Next the chin strap 440, 445 or securing means 430 is connected to the inventive head pre-formed absorbent wound dressing 400 again by way of non-limiting example of RF welding. The chin strap 440, 445 as shown in FIGS. 25, 27A-27B and 28 includes a sliding portion 445 having two apertures or openings 442, 442 through which the chin strap 440 is inserted and allows in this configuration for the sliding portion 445 to be moved along the chin strap 440 for placement by the user at the chin of the individual wearing the inventive head pre-formed absorbent wound dressing 400, which takes into account various sizes of heads and faces by individuals who would have the inventive head pre-formed absorbent wound dressing 400 placed upon them. The opening for the face has a certain shape in the embodiments shown in FIGS. 25-28 but the shape can be changed so that more or less of the forehead is covered by the inventive head pre-formed absorbent wound dressing 400 or the sides can be changed so that more or less of the cheeks are visible and covered or less cove red by the inventive head pre-formed absorbent wound dressing 400. While not shown it is envisioned that an embodiment can be used to cover the face with opening or apertures for the nose, mouth and/or eyes and then wound site or sites on the face can be covered to control bleeding and remove exudate.

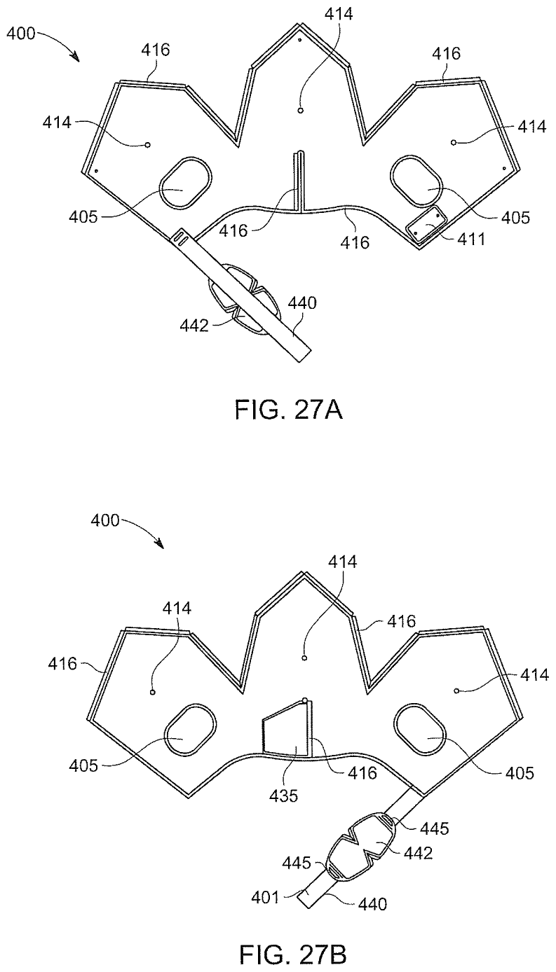

In the embodiments shown in FIG. 25, the chin strap 440 is connected on one end to the inventive head pre-formed absorbent wound dressing 400 via RF welding and the other end has a loop portion (not shown in FIG. 25 but shown as 411 on FIGS. 27A and 28) on the underside of the film or polymer chin strap 440 that can be secured or connected to the hook 411 welded onto the opposite face side of the inventive head pre-formed absorbent wound dressing 400. In another embodiment as shown in FIG. 26 the fastening or securing means 430 is two fasteners or tabs or chin straps 446, 448 which can be connected on one end each to the inventive head pre-formed absorbent wound dressing 400 via by way of non-limiting example welding and the other side by any known fastening means such as adhesives or hook and loop portions. In this embodiment there are two chin straps 446, 448 but there could be only one or three or more as needed for the configuration. Not shown but possible is a underside portion that is hook or loop capable of connection to a corresponding securing means such as a hook or loop portion such as the loop portion 411 shown in FIGS. 27A and 28. Other connecting or securing or fastening means may be employed such as snaps, buckles, connectors and combinations thereof.

In use the emergency, clinical or medical personnel places the inventive head pre-formed absorbent wound dressing 400 on the head with the ears placed with the apertures or openings 405, 405. Next the user can either attached the chin strap 440, 446, 448 first or adjust the face opening via the adjusting means or fastener 435. If the user chooses to first connect the chin strap 440, 446, 448 it can be connected in these embodiments via the hook on the underside of the chin strap 440, 446, 448 to the loop portion 411 and moved up and down to secure the inventive head pre-formed absorbent wound dressing 400 on the chin and head of the patient or individual needing bleeding control and wound management. The user can then adjust the fit and tightness at the face near the forehead in these embodiments through the adjusting means such as the flap or fastener 435 using the underside loop layer 431 connected to the hook portion 401. Through this adjustment the opening for the face is tightened to prevent blood and exudate from going onto the face. This adjusting means is only one of various possibilities and others may be employed to better fit the inventive head pre-formed absorbent wound dressing 400 onto the head when in use. The inventive head pre-formed absorbent wound dressing 400 may also be placed on the head over an optional gauze 50 impregnated with medication, anti-bacterial; coatings, one or more coatings, silver or zinc oxide already on the wound site as well as an elevation member or bolster (not shown but various embodiments are disclosed in co-owned application Serial No. PCT/US23/25642 such as a circle or oval to be placed on the wound site to assist in treatment and prevention of bed sores on the head as well as a crescent shape for use around the ear 705 and other shapes may be employed. The advantages of the inventive head pre-formed absorbent wound dressing 400 is that it has the pre-formed shape of the head for quick and easy application onto the head during an emergency situation or by any clinical or medical professional as well as the user himself/herself given its pre-formed shape. Further, the user can adjust the shape of the face making it tighter as needed, and also the user can remove the chins strap or fasteners 440, 446, 448 so that one can view the wound site and check for healing as well as infection, and the individual wearing it can take a shower or go about daily living then place the inventive head pre-formed absorbent wound dressing 400 back on the head using the chin strap or fasteners 440, 446, 448. Other advantages are contemplated by the inventive head pre-formed absorbent wound dressing 400 as well as other shapes and securing or shaping means.

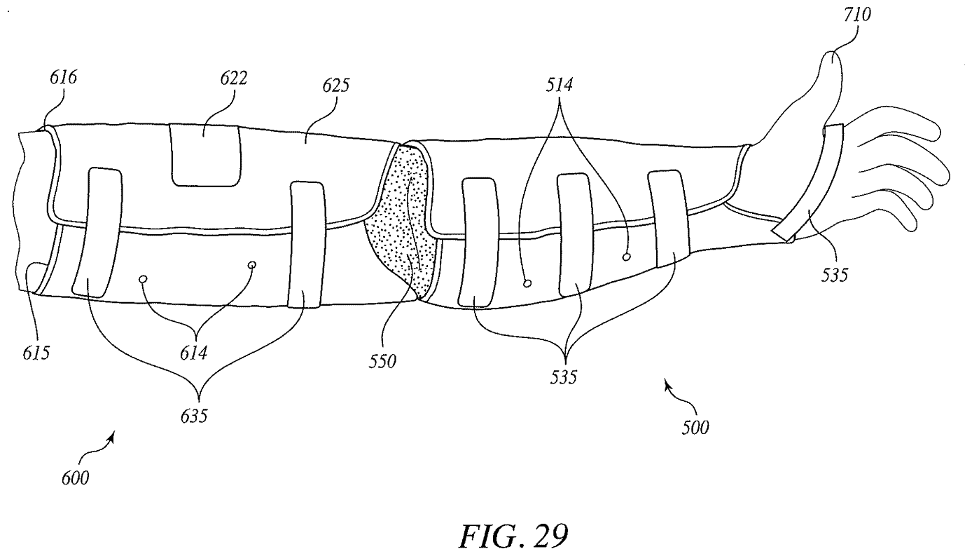

In yet another embodiment of the present invention a lower arm pre-formed absorbent wound dressing 500 and upper arm pre-formed absorbent wound dressing 600 are shown in FIG. 29. In the lower arm pre-formed absorbent wound dressing 500 there are four fasteners or tabs 535 for securing the lower arm pre-formed absorbent wound dressing 500 to the arm and wrist area with the fastener or tab 535 on the distal portion wrapping around the thumb 710, though other securing means may be employed. The distal portion of the inventive lower arm pre-formed absorbent wound dressing 500 is shaped to fit over the wrist and ends before the elbow. The other embodiment of the upper arm pre-formed absorbent wound dressing 600 include two fasteners or tabs 635, 735 as well as a shaping or support means 622, by way of non-liming example a plastic support 622 to shape for an upper arm and bicep area above the elbow and below the shoulder. In this embodiment as shown in FIG. 29 an elastic member 550 is included over the elbow area but there could also be a joining or connecting means (not shown) to connect with the lower arm pre-formed absorbent wound dressing 500 with the upper arm pre-formed absorbent wound dressing 600. The number of fasteners or tabs 535, 635 in each embodiment may be increased or decreased as needed for the selected or target body part. In use either the lower arm pre-formed absorbent wound dressing 500 or the upper arm pre-formed absorbent wound dressing 600 may be placed on the arm over a wound dressing including silver or zinc oxide 50 and a bolter or elevation member 75 and under a therapeutic compression apparatus or even a dialysis compression apparatus as disclosed in co-owned application PCT/US22/024121 and U.S. patent application Ser. No. 18/285,853 entitled “Arm Therapeutic Compression System, Apparatus and Methods of Use”, the entire contents of which are incorporated herein.

The inventive pre-formed absorbent wound dressing 100, 200, 300, 400, 500, 600 has many advantages in that it may absorb the execute from the ulcer or wound site while cushioning the wound area and surrounding limb such as the leg, foot, arm or head and based on its shape and openings (111, 113, 114, 116, 117, 211, 214, 216, 305, 314, 316, 405) is more secure on the leg, foot, head, arm and other body parts when in use, particularly when walking as well as on the arm or head. The outer layer 125, 225, 325, 425, 525, 625 does not allow the excretion, pus, blood or the like from harming the user's socks, clothes, bed linens, couch or the like as it is non-absorbent or less-absorbent than the inner layer 115, 215, 315, 415, 515, 615 and the central or middle absorbent layer 120, 220, 320, 420, 520, 620. The securing means such as the fasteners and tabs 135, 235, 335, 435, 535, 635 support the shape of the target limb such as the lower leg, foot or arm so it does not fall down on the ankle or bunch under or on top of the foot when the user walks or down the wrist of the arm or even moving around the head and neck. Currently, a user would have gauze or rolls of surgical tape and wrap these known wound dressings around the leg, foot, arm or head which is cumbersome, uneven, and has gaps on the calf, ankle, toes, arch, sole, foot, knee, arm, elbow, wrist, shoulder or neck and head. The inventive pre-formed absorbent wound dressing 100, 200, 300, 400, 500, 600 solves these problems and is a secure wound dressing whether used alone or in a system with a therapeutic compression apparatus 1000, and/or the wound dressing 50 and/or the elevation apparatus or bolster 75.

Optionally, the inventive pre-formed absorbent wound dressing 100, 200, 300, 400, 500, 600 may include a drain port in fluid communication with either the inner layer 115, 215, 315, 415, 515, 615 or middle absorbent layer 120, 220, 320, 420, 520, 620 to drain excessive exudate, serum or blood from the ulcer or wound site. Optionally another layer may be employed that is filled with a filler material such as, for example, a gel that can be, for example, a silicon gel or polytetrafluoroethylene (PTFE). The gel-like substance may have a flow characteristic that allows the inventive pre-formed absorbent wound dressing 100, 200, 300, 400, 500, 600 conform to the contour of the wound site. Optionally, the inventive pre-formed absorbent wound dressing 100, 200, 300, 400, 500, 600 may include an indicator to show when the inventive pre-formed absorbent wound dressing 100, 200, 300, 400, 500, 600 has a level of exudate within and should be changed, or any other indicators such as moisture, filled capacity, temperature of the target limb, pulse rate at the target limb or a pressure measurement device having a digital readout for the target limb such as the ankle or foot or leg or knee or the like of the individual user. Non-limiting examples of indicators are moisture level sensors or pressure sensors or measurements devices include a micro-array of pressure sensors at different sites including the target ulcer or wound site and sites around the target ulcer or wounds site on the foot or leg or other limbs. Further, the pre-formed absorbent wound dressing 100, 200, 300, 400, 500, 600 may include sensors regarding CO2 levels, temperature, pulse rate, and combinations thereof.

The inventive compression system includes the inventive pre-formed absorbent wound dressing 100, 200, 300, 400, 500, 600 with a therapeutic compression apparatus 1000 (as shown in FIG. 30), over the inventive lower leg pre-formed absorbent wound dressing 100 which is over both an optional gauze wound dressing 50 impregnated with medication, anti-bacterial coating, one or more coatings, silver, or zinc oxide and a bolter or elevation apparatus 75 (shown as a crescent along the ankle 630 in FIG. 30 though other shapes may be contemplated and employed as well as more than one bolster or elevation apparatus used at the same time on different locations of the selected or target body part). Examples of the therapeutic compression apparatus include those co-owned by Applicant known as the AeroDVX, Aero-Wrap and Sun Wrap and those disclosed in USPTO application Ser. No. 17/608,323; Ser. No. 63/425,183 entitled “Inelastic Therapeutic Compression Apparatus, System And Methods Of Use”; U.S. Pat. Nos. 9,033,906; 7,967,766; 7,559,908; Ser. No. 13/444,600; and Ser. No. 16/328,718, which are incorporated in their entirety herein, as well as the previously cited arm therapeutic apparatus for use with the inventive lower arm and upper arm pre-formed absorbent wound dressings 500, 600. In some embodiments the therapeutic compression apparatus 1000 is capable of compression via an inflation means which may be a manual hand pump, foot pump, mechanical pump, electrical pump, battery-operated pump, static pump, intermittent pump, varying pump, automatic pump, pneumatic pump, negative pressure pump, suction pump or vacuum, pulsing pump, or any combination thereof or any other known or developed source of inflation so as to provide a certain pressure within the therapeutic compression apparatus so to provide compression in use by the patient. In some embodiments the inflation means is connected to the therapeutic compression apparatus by a hose or tube. In some embodiments of the inflation means is connected to a retaining means such as a belt or wrap to be worn by the user.

The inventive pre-formed absorbent wound dressing 100, 200, 300, 400, 500, 600 has many advantages in that it may absorb the execute from the wound while cushioning the wound area and surrounding leg or foot and based on its pre-formed shape it hugs or is more secure on the leg and foot when in use, particularly when walking, both over the ankle and below the knee area. The outer layer 125, 225, 325 425, 525, 625 does not allow the excretion, pus, blood or the like from harming the user's clothes, bed linens, couch or the like as it is non-absorbent or less-absorbent than the absorbent inner layer 115, 215, 315, 415, 515, 615. The shaping means such as the wires, plastic or elastic 122, 124, 126 support the shape of the limb so that the inventive lower leg pre-formed absorbent wound dressing 100 does not fall down on their leg when the user walks. The shape configuration of the foot version of the inventive pre-formed absorbent wound dressing 200 including the heel opening or aperture 211 and the shape or configuration of the tabs 235 prevent or reduce the inventive pre-formed foot absorbent wound dressing 200 from falling down from the ankle area 630 of the foot when the user is walking, both barefoot and when the inventive foot pre-formed absorbent wound dressing 200 is within a sock or shoe. In addition, the inventive foot and toe spacer pre-formed absorbent wounds dressing 300 is shaped for easy application to the bony foot and toes 610 by the user or patient as well as emergency, clinical and/or medical personnel. Currently, a user would have gauze or rolls of surgical tape and wrap these known wound dressings around the leg, in between the toes or foot, which is cumbersome, uneven, and has gaps on the leg at the calf, knee 650, ankle 630, toes 610 and foot. The inventive pre-formed absorbent wound dressings 100, 200, 300, 400, 500, 600 solve these problems and is a secure wound dressing whether used alone or in a system with a therapeutic compression apparatus 1000. The inventive thigh pre-formed absorbent wound dressing (not shown) can also be used over a bolster and below a therapeutic compression apparatus such as disclosed in co-owned PCT applications cited above. The advantages of the inventive lower arm and upper arm inventive pre-formed absorbent wounds dressings 500, 600 are the same as discussed above regarding the lower leg and foot 100, 200, 300.

Also possible are other shapes or indents and apertures within the inventive pre-formed absorbent wounds dressings for a specific known incision or surgical wound such as in knee replacement or hip replacements when the target limb for the inventive pre-formed absorbent wound dressing a knee, full leg, torso or hip area, as well as during or after dialysis on the arm or after a mastectomy such as disclosed in co-owned application PCT/US20/057224 entitled “Therapeutic Compression System and Methods of Use”, the entirety which is incorporated herein. Other limbs may also be secured with the inventive pre-formed absorbent wound dressing 100 wherein the shape of the inventive pre-formed absorbent wound dressing 100 is a cone, or other shapes configured for the target limb.

The inventive pre-formed absorbent wound dressings solve the problem of irregular bandaging or wound treatment. Typically a nurse or care giver will current wrap gauze around leg such that some portions of the wrapped gauze are thicker and other portions thinner resulting in irritation on the leg or wound area as well as leakage of the exudate from the wound site. The user themselves may not be able to self-apply and even a skilled caretaker or medical professional may wrap such a wound dressing either too tight resulting in circulation problems or too loose with leakage and gaps. Currently there is no consistency in applying absorbent wound dressing over a wound site on certain limbs. The inventive pre-formed absorbent wound dressings 100, 200, 300, 400, 500, 600 solves this problem by having a pre-formed shaped configured for the selected or target body part. The inventive pre-formed absorbent wound dressings 100, 200, 300, 400, 500, 600 includes a certain level of shape and stiffness, such as via a set of wires or plastic or other materials such that the inventive pre-formed absorbent wound dressing 100, 200, 300, 400, 500, 600 conforms to the shape of the target limb. The inventive pre-formed absorbent wound dressing 100, 200, 300, 400, 500, 600 has better fit, hugs or secures the absorbent wound dressing to the target limb while also not allowing leaks of exudates, puss, blood or the like with a non-absorbent outer layer. These and other advantages can be found with the use of the inventive pre-formed absorbent wound dressing 100, 200, 300, 400, 500, 600.

The inventive pre-formed absorbent wound dressing 100, 200, 300, 400, 500, 600 make drainage management for wounds and exudate easier for both the emergency, clinical and medical personnel as well as the individual wearing and using the inventive pre-formed absorbent wound dressing 100, 200, 300, 400, 500, 600. The shaping or support means such as the wires, plastic bands or elastic 122, 124, 126 make it easy to apply and to open to check on the wound site and close it up again multiple times. Notably the user or patient can self-manage their care in between appointments with clinical or medical professionals.

The inventive wound dressing compression system including the inventive pre-formed absorbent wound dressing 100, 200, 300, 400, 500, 600 also reduces the problem of lack of mobility in that the patient can walk about and go to work, school, recreational activities. The inventive system where including the therapeutic compression apparatus 1000 also includes inflation means which is not tethered to a wall outlet and instead is a main pump which is configured to either apply constant static pressure at one pressure level, or constant at static pressure at a choice of different pressure levels, or intermittent pressure at one level, or intermittent pressure at multiple pressure levels, or a choice of either constant static pressure level and intermittent pressure levels. The ability to switch between pressure levels and/or switch between constant static pressure and intermittent pressure promotes a more effective treatment for CVI, DVT and/or lymphedema and other treatments. Another embodiment (not shown) may include a variety of sensors so that the pump on its own may adjust the pressure level or switch from intermittent pressure level (which the patient is sitting or the kg is elevated) to constant static pressure (when the patient is walking or running). Such sensors may be connected to a database accessible by a medical provider which could remotely adjust the pressure levels or status change from intermittent to constant or the reverse.

The inventive system may be used as a prophylaxis or as part of a treatment plan which is easy for the patient to use at home or work (outside a hospital setting or with the aid of a medically trained professional as noted above) which is ambulatory so that the patient can walk and return to life activities. The inventive system can be used as a prophylaxis for wound care and/or swelling in any body part. The inventive system can also be used in pre-operative and post-operative treatments for many different surgeries including but not limited to knee surgeries, hip surgeries, TKR, KRA, n-m, HRA, sclerotherapy and many other surgical procedures regarding other libs or body parts which have exudating wounds as well as an increased risk of CVI and/or DVT. The inventive system may be used to prevent, reduce or even treat DVT and the system is practical to use, mobile and easy to be administered by the patient post-operative total knee replacement of any other knee, hip or leg surgery. Further the inventive system could also be used in HPIPC treatment and this inventive system can be easily administered by a patient in the home setting as well as a rehabilitation setting or nursing home setting.

The inventive pre-formed absorbent wound dressing 100, 200, 300, 400, 500, 600 alone or in combination with the inventive system democratizes the wound health care in that the user can remove the wound dressing to engage in everyday activities such as bathing and does not need to travel to a medical professional every few days for removal of wound dressings and replacement. It allows the user to self-apply the inventive pre-formed absorbent wound dressing 100, 200, 300, 400, 500, 600. Further, the user saves money in travel costs and co-pays as well as time with reduced visits to the medical professional. Notably the inventive pre-formed absorbent wound dressing 100, 200, 300, 400, 500, 600 is reusable saving money and time. The inventive pre-formed absorbent wound dressing 100, 200, 300, 400, 500, 600 provides measurable air to the target ulcer or wound site, a compression level that is maintainable throughout the treatment plan when employing the therapeutic compression apparatus, is capable of self-application by the patient including being able to remove the inventive pre-formed absorbent wound dressing 100, 200, 300, 400, 500, 600 and reuse it, and these needs are all met by the inventive pre-formed absorbent wound dressing 100, 200, 300, 400, 500, 600 whether alone or in combination with the system including a therapeutic compression apparatus 200, 300, 500, and/or a wound dressing 50 and/or an elevation apparatus or bolster 75. Notably, anyone can properly place the inventive pre-formed absorbent wound dressing 100, 200, 300, 400, 500, 600 on the user in a more effective way than known conventional compression bandages, which exudating the wound site or ulcer and assisting in wound healing, and if using the optional bolster 75 or therapeutic compression apparatus 200, 300, 500 further reduce edema and potential bed sores or ulcers.

This inventive pre-formed absorbent wound dressings 100, 200, 300, 400, 500, 600 ease of reapplication directly correlates to compliance by the patient with the treatment plan. The inventive pre-formed absorbent wound dressing 100, 200, 300, 400, 500, 600 assist in healing and reduction of complications by providing a multi-layer design which is comfortable, non-stick, absorbent, and has an outer backing layer 135 that prevent strike through but allow vapor transfer so not occlusive to the target foot ulcer or wound site by reducing build up in Co2 or bacteria growth. The inventive pre-formed absorbent wound dressing 100, 200, 300, 400, 500, 600 provides measurable air to the wound site.

While the subject invention of the present disclosure has been described with respect to preferred and exemplary embodiments, those skilled in the art will readily appreciate that various changes and/or modifications can be made to the invention without departing from the spirit or scope of the invention as described herein. There have been described and illustrated herein several embodiments of an pre-formed absorbent wound dressing 100, 200, 300, 400, 500, 600 and a method of applying and securing same onto a target limb of a user as well as a compression system including the inventive pre-formed absorbent wound dressings 100, 200, 300, 400, 500 600. While particular embodiments of the invention have been described, it is not intended that the invention be limited thereto, as it is intended that the invention be as broad in scope as the art will allow and that the specification be read likewise. Thus, while particular shapes and sizes of pre-formed absorbent wound dressings and their components have been disclosed, it will be appreciated that other shapes, sizes, and attachment means may be used as well. It will also be understood that while adhesive means have been disclosed for helping to secure the pre-formed absorbent wound dressing 100, 200, 300, 400, 500, 600 to the selected or target body part and other types of attachments such as hooks, snaps, or wraps may be used. In addition, it will be appreciated that certain materials have been disclosed for the various hydrophilic, absorbent, wicking, hydrophobic and hydroconductive layers while other materials known in the art may be used. It will therefore be appreciated by those skilled in the art that yet other modifications could be made to the provided invention without deviating from its spirit and scope as claimed.

We claim:

1. A pre-formed absorbent wound dressing for use on a selected body part, comprising:
an inner porous layer having contact with a wound site;
a middle absorbent layer configured to remove and store exudate from the wound site;
an outer waterproof vapor transfer layer;
a plurality of connections sealing the inner porous layer and outer waterproof vapor transfer layer together over the middle absorbent layer; and
a securing means for securing the pre-formed absorbent wound dressing to the selected body part,
wherein the pre-formed absorbent wound dressing is configured in a shape for placement on the selected body part.

2. The pre-formed absorbent wound dressing of claim 1 wherein the selected body part is selected from the group of foot, ankle, leg, calf, knee, thigh, hip, torso, chest, shoulder, arm, elbow, wrist, hand, neck, and head.

3. The pre-formed absorbent wound dressing of claim 1 wherein the inner porous layer is a hydrophilic layer or a hydroconductive layer.

4. The pre-formed absorbent wound dressing of claim 1 wherein the middle absorbent layer is selected form the group consisting of cotton, pulp, cellulose, cellulose derivatives, polymers, high molecular water absorbent polymers, thermoplastic resin, cellulosic fiber, acrylic (salt) polymer, foam, woven or nonwoven material selected from the group consisting of rayon, polyester, polyurethane, polyolefin, orlon, hydrogel polymeric material, and combinations thereof.

5. The pre-formed absorbent wound dressing of claim 1 wherein the inner porous layer or middle absorbent layer further includes a wicking layer.

6. The pre-formed absorbent wound dressing of claim 1 wherein the outer waterproof vapor transfer layer is moisture permeable and liquid impermeable.

7. The pre-formed absorbent wound dressing of claim 1 wherein the outer waterproof vapor transfer layer is hydrophobic and is selected from the group consisting of polymers, woven and non-woven materials, films, laminates and combinations thereof.

8. The pre-formed absorbent wound dressing of claim 1 wherein the outer waterproof vapor transfer layer further comprises a hydrochromatic ink.

9. The pre-formed absorbent wound dressing of claim 1 wherein the inner porous layer further includes medications, anti-bacterial coatings, zinc oxide, silver or combination thereof.

10. The pre-formed absorbent wound dressing of claim 1 wherein the plurality of connections seals all three layers together on a plurality of edges of said three layers.

11. The pre-formed absorbent wound dressing of claim 1 wherein the securing means is selected from the group of adhesives, hook and loop, straps, buckles, snaps, and combinations thereof.

12. The pre-formed absorbent wound dressing of claim 1 wherein the securing means is an adhesive capable of sealing, releasing and sealing multiple times.

13. The pre-formed absorbent wound dressing of claim 12 wherein the securing means includes a tab having a distal portion without any adhesive configured to allow the user to grip the securing means for opening and closing.

14. The pre-formed absorbent wound dressing of claim 1 further comprising a support means configured so the pre-formed absorbent wound dressing is in a shape for placement on the selected body part, wherein the support means is selected from the group consisting of plastic, polymers, wires, elastic, and combinations thereof.

15. The pre-formed absorbent wound dressing of claim 14 wherein the selected body part is a lower leg from an ankle portion to a knee portion, and the pre-formed absorbent wound dressing is configured in the shape of lower leg to be placed above the ankle including an opening for the back of the ankle and below the knee, wherein the support means are wires, plastic or elastic and are located along the calf area.

16. The pre-formed absorbent wound dressing of claim 1 wherein the selected body part is a foot and the pre-formed absorbent wound dressing is configured including an opening for a heel of the foot and capable of being wrapped over an arch of the foot leaving a toe area of the foot unwrapped.

17. The pre-formed absorbent wound dressing of claim 1 wherein the selected body part is a foot and the pre-formed absorbent wound dressing is configured including a plurality of openings for a number of toes of the foot and capable of being wrapped over either an arch of the foot or a sole of the foot.

18. The pre-formed absorbent wound dressing of claim 1 wherein the selected body part is a head and the pre-formed absorbent wound dressing is configured including a plurality of openings for a face and two ears of a head and further comprising an adjusting means configured to adjust the opening at the face and wherein the securing means is configured to be placed on a chin of the head and the adjusting means includes a hook portion and a loop portion configured for the user to adjust the open face portion at a forehead area of the head.

19. The pre-formed absorbent wound dressing of claim 18 wherein the securing means further includes a chin support configured to slide along the securing means for placing the chin support at the widest point of the chin on the face when in use on a head.

20. The pre-formed absorbent wound dressing of claim 18 wherein the adjusting means is selected form the group consisting of adhesives, hook and loop, straps, buckles, snaps, and combinations thereof.

21. The pre-formed absorbent wound dressing of claim 20 wherein the adjusting means is comprised of a slit in the top portion of the pre-formed absorbent wound dressing located over the head, a hook portion on top portion of the head and a loop portion under a flap.

22. The pre-formed absorbent wound dressing of claim 1 wherein the selected body part is a lower arm and the pre-formed absorbent wound dressing is configured including a securing means for securing around a thumb of the user at a wrist location.

23. The pre-formed absorbent wound dressing of claim 1 wherein the selected body part is an upper arm and the pre-formed absorbent wound dressing is configured including a shaping means for shaping the pre-formed absorbent wound dressing to confirm to the shape of an upper arm as well as securing the pre-formed absorbent wound dressing at the upper arm and bicep area.

24. A wound dressing and compression system for use on a body part, comprising:
(a) a pre-formed absorbent wound dressing for use on a selected body part, comprising (i) an inner porous layer having contact with a wound site, (ii) a middle absorbent layer configured to remove and store exudate from the wound site, (iii) an outer waterproof vapor transfer layer, (iv) a plurality of connections sealing the inner porous layer and outer waterproof vapor transfer layer together over the middle absorbent layer, and (v) a securing means for securing the pre-formed absorbent wound dressing to the selected body part, wherein the pre-formed absorbent wound dressing is configured in a shape for placement on the selected body part; and
(b) a therapeutic compression garment comprising (i) a bladder configured to apply compression on a selected body part and having an inflation port, (ii) a securing means for use on the selected body party, and (iii) an inflation means, wherein the therapeutic compression garment is configured in a shape for placement on the selected body part.

25. The wound dressing and compression system of claim 24 further including an elevation member having a foam portion and a bladder portion configured to elevate a selected body part.

26. The wound dressing and compression system of claim 24 wherein the elevation member is in the shape of a circle, oval, crescent, dog bone, rectangle, square, semi-circle, and any other known shape.

27. The wound dressing and compression system of claim 24 further including a second dressing including a coating selected from the group of medication, anti-bacterial materials, silver, zinc oxide or combinations thereof.
